(19) United States
(12) Patent Application Publication
(10) Pub. No.: US 2009/0138285 A1
(43) Pub. Date: May 28, 2009

(54) Health Promotion Outreach System

(76) Inventor: Thomas D. Denberg, Denver, CO (US)

Correspondence Address: GREENLEE WINNER AND SULLIVAN P C, 4875 PEARL EAST CIRCLE, SUITE 200, BOULDER, CO, 80301, US

(21) Appl. No.: 12/277,177

(22) Filed: Nov. 24, 2008

Related U.S. Application Data
(60) Provisional application No. 60/990,200, filed on Nov. 26, 2007.

(51) Int. Cl. G06Q 50/00; G06F 17/30; G06Q 40/00; G06Q 10/00; G06F 7/06; A61B 5/00

(52) U.S. Cl. 705/3; 707/104.1; 707/4; 600/301; 707/E17.005; 707/E17.014

(57) ABSTRACT

Patient receipt of recommended preventive and chronic disease care is suboptimal, partly because of poorly organized clinical information and time-limited and sporadic appointments with medical providers. To overcome these problems, we provide a Health Promotion Outreach System (HPOS) based on electronic data queries, active patient-centered outreach, and minimizing patient barriers to adherence. HPOS has many advantages over traditional preventive care delivery systems and chronic disease management programs, and helps promote the type of clinical practice change contemplated by the chronic care model.

### CROSS-REFERENCE TO RELATED APPLICATIONS

This application claims priority to U.S. Provisional Patent Application Ser. No. 60/990,200, filed Nov. 26, 2007, which is incorporated herein by reference to the extent not inconsistent herewith.

### BACKGROUND

Delivery of preventive and chronic disease care in ambulatory settings is often suboptimal (Kabcenell, Langley, & Hupke, 2006; Ostbye et al., 2005; Yarnall, Pollak, Ostbye, Krause, & Michener, 2003). Time-limited clinical encounters are often too brief for medical providers to review and arrange for all recommended services. Patients have competing agendas during clinic visits, usually related to acute complaints. Large numbers of patients prefer not to see medical providers unless they have an acute complaint. Finally, medical providers do not always remember to discuss and arrange for preventive and chronic disease services and are hampered by poorly-organized clinical data and a lack of automated reminders.

Although decision support and electronic, point-of-care prompting in the medical provider's office can improve guideline-based care (Schmittdiel et al., 2004), these mechanisms do not improve outcomes among patients who fail to make clinic appointments.

HMO-exclusive medical groups operating within integrated health systems (e.g. Kaiser Permanente and Group Health Cooperative) and the Veterans Affairs integrated health network have set a high bar for delivering comprehensive care, tracking outcomes, and carrying out continuous quality improvement (Kerr et al., 2004; Lomas, 2003; Stevens, 1998). Central to these efforts are well-developed health information technology (HIT) infrastructures that provide clinical decision support, registry-building capacity, and electronic communication to coordinate care among members of a health care team. Also essential is a strong commitment to these activities by institutional leadership.

In comparison with such high-functioning integrated health systems, other kinds of medical groups operate within smaller hospital and clinic systems, have correspondingly smaller budgets, less HIT infrastructure and quality improvement (QI) expertise, and have been shaped by a markedly different set of market pressures and incentives.

All publications referred to herein are incorporated herein by reference to the extent not inconsistent herewith for purposes of written description and enablement.

### SUMMARY

The Health Promotion Outreach System (HPOS) described herein increases access to and enhances the delivery of guideline-based care by communicating with patients outside of clinical settings. HPOS enables outreach to patients to facilitate receipt of services reflective of health care quality as defined by organizations such as the National Committee for Quality Assurance (NCQA), the American Diabetes Association, and the United States Preventive Services Task Force.

This system allows healthcare organizations that contract with various payers to deliver high-quality, evidence-based preventive and chronic disease care more effectively to their patients without incurring excessive costs. The method promotes patient convenience and satisfaction in a competitive market environment, improves pay-for-performance measures for regulatory purposes and public reporting, and increases the utilization of medical services, thereby increasing revenue and attracting new patients and payers.

The system dramatically improves patient care within a wide variety of health care organizations. Oriented around electronic data queries, active patient-centered outreach, and minimizing barriers to adherence, the system is designed to reduce the number of patients who “fall through the cracks” when it comes to receiving guideline-based care. We also describe advantages of HPOS over traditional preventive care delivery systems and chronic disease management programs, and explain how HPOS complements and helps to promote the type of clinical practice change contemplated by the chronic care model. Finally, we briefly describe some of our initial experience with HPOS at the University of Colorado Hospital (UCH).

HPOS has the following core characteristics:
HPOS is based within and staffed by employees of a health care organization in which an ambulatory population of patients receive their care. Alternatively, HPOS can be provided as a third-party service to a health care organization that cares for an ambulatory patient population. HPOS helps to realize a patient-centered medical home model by making use of up-to-date clinical information, streamlining access to clinical services, and facilitating timely communication between providers and patients.
HPOS uses database queries of electronic administrative and/or medical record data to identify patients eligible for guideline-concordant services.
HPOS employs written followed by telephonic patient outreach in order to maximize the number of patients who are notified, educated about, and subsequently receive guideline-based care. Outreach via letter and telephone educates patients about their medical conditions and provides individualized recommendations for chronic disease management, thus supplementing the information that patients receive during face-to-face clinician visits. After receiving written correspondence, patients are invited to contact a call center to arrange for recommended services. If patients do not make in-calls after a specified period of time, and if they have not opted out of the service, coordinators make out-calls to patients, thereby reaching those who are interested but have forgotten or not “gotten around” to calling.
While patients are on the phone, outreach coordinators schedule provider visits as needed and, whenever possible, also directly schedule laboratory tests and subspecialty consultations as indicated by guidelines. Such one-step scheduling maximizes patient convenience and adherence, minimizes barriers to access such services, and increases the chance of successfully completing the recommended actions.
Providers agree prior to the implementation of particular services that this automated protocol is medically appropriate. They authorize services through clinic protocols and/or by means of confirmatory electronic or written signatures on individual patient referrals and requisitions. The results of all tests and services are then communicated directly to authorizing providers so that they can provide appropriate follow-up. This workflow supports provider autonomy and minimizes inappropriate HPOS interventions.
The results of patient outreach, including information obtained by telephone about patients' clinical status, concerns, and preferences, are documented in the medical record for clinical continuity.
To carry out the tasks described above, registered nurse (RN)-level staffing is unnecessary; instead, we have used lower-cost outreach coordinators with BA-level training.
HPOS is an adjunctive and supportive system. In face-to-face encounters, providers care for patients as they always have. HPOS imposes minimal additional burden on medical providers and does not supersede established clinic routines. In fact, HPOS can improve the quality and efficiency of in-person care by, for example, helping patients to complete laboratory testing several days before clinic appointments.
As a “clinical extender,” the HPOS helps medical providers identify, reach out to, communicate with, and provide higher quality care to their patients. Because the HPOS is able to carry out routine tasks that do not require the direct involvement of physicians, more time is made available for medical providers to focus on the complexities of patient care during actual face-to-face encounters. In addition, patient access to providers is improved when clinic slots are used more judiciously.
Because patients are contacted on a recurring basis, registry maintenance and accuracy are improved. For example, when patients move their addresses and phone numbers are updated, and when they no longer receive care within the health system, they can be removed from the registry. This is important because the lack of timely and accurate patient information commonly limits the effectiveness of registry-based interventions.85
Several advantages are associated with mailing tailored letters to patients prior to telephone contact. First, a letter “signed” by a patient's PCP make clear that the PCP is “keeping track” of the patient's health and medical needs between visits and allow an established therapeutic alliance between physician and patient to be harnessed in order to promote adherence to guideline-based care. Second, patients who receive personalized clinical summaries of their disease status (e.g. “Diabetes Score Cards”) accompanied by written recommendations for care and educational materials are able to consider this information carefully, formulate questions, and check their calendars before speaking with an HPOS outreach coordinator. This supports patient activation and increases the efficiency of telephone communication when it occurs. Third, because letters encourage patients to contact the HPOS call center directly, cost savings accrue when outreach coordinators do not have to initiate multiple call attempts to all patients. Fourth, the ability to schedule multiple clinical services at a single point in time necessitates a live human operator. By employing HPOS outreach coordinators to carry out scheduling tasks, patients can avoid the responsibility and inconvenience of calling separate scheduling units themselves, waiting on hold, and leaving voice messages requesting call backs. Because traditional scheduling units are not needed with HPOS, the unavoidable costs of telephone-based scheduling are shifted to an HPOS program that carries out these functions more efficiently and is able to achieve higher levels of patient satisfaction. Finally, it is only live operators who can answer questions and handle the highly variable and often complex scheduling challenges inherent to chronic disease management. In summary, the combination of tailored letters and multiple, live telephone contact attempts is a logical, compelling, and fundamental feature of the HPOS recall program we have developed.
HPOS is proactive, patient-centered, systems-based, and population- and disease-focused. For diabetes and other chronic conditions, HPOS establishes key elements of the chronic care model (CCM) in ambulatory settings. It is physically based in a provider organization and well-integrated into primary care practices. It therefore has many advantages over commercial disease management programs. Alleviating some of the production pressures placed on ambulatory care providers by assuming responsibilities that do not require advanced clinical training (e.g. phone outreach), HPOS allows providers to give greater attention to the complexities of diagnosing and treating disease. The HPOS concept is scalable and adaptable to many kinds of integrated healthcare systems, and is a useful tool for improving quality of care and performance measures. It increases patient satisfaction and allegiance to a health care organization by making best practice transparent and customized to the patient and by eliminating barriers to receiving such care. Finally, because HPOS harnesses advances in information technology and the values of patient-centered medicine, it helps to transform traditional systems of care into more effective planned care for routine screening chronic disease management.

### BRIEF DESCRIPTION OF THE DRAWINGS

FIG. 1 is a flow sheet depicting the flow of information in the system.

FIG. 2 is a screenshot of the Logon screen for the tracking algorithm.

FIG. 3 is a screenshot of the Main Menu screen.

FIG. 4 is a screenshot of the Patient Eligibility screen.

FIG. 5 is a screenshot of the Patient Eligibility screen displaying a subset of eligible patients.

FIG. 6 is a screenshot of the Mailing Menu screen.

FIG. 7 is a screenshot of the Process Received Postcards screen.

FIG. 8 is a screenshot of the Select Active Test screen.

FIG. 9 is a screenshot of the Patients Eligible for Telephone Contact screen.

FIG. 10 is a screenshot of the Patient Information and Telephone Contacts screen.

FIG. 11 is a screenshot of the Scheduling Management screen.

FIG. 12 is a screenshot of the Patient Information for Scheduling screen.

FIG. 13 is a screenshot of the Test Disposition screen.

FIG. 14 is a screenshot of the Patient Test Disposition screen.

FIG. 15 is a screenshot of the Test Report screen.

FIG. 16 is a screenshot of the Reporting Options screen.

FIG. 17 is a screenshot of a text report generated by selection of options on the reporting options screen of FIG. 16.

FIG. 18 is a screenshot of the Security Options menu screen.

FIG. 19 is a screenshot of the Change Password screen.

FIG. 20 is a screenshot of a User and Group Account Management screen.

FIG. 21 is a screenshot of the Database Menu Management screen.

FIG. 22 is a screenshot of the Import New Patients and Tests. screen.

FIG. 23 is a screenshot of a selection box that appears when DM is selected on the Import New Patients screen of FIG. 22.

FIG. 24 is a screenshot of the Close Tests screen.

FIG. 25 is a screenshot of the Physician Management screen.

FIG. 26 is a screenshot of the System Information Management screen.

FIG. 27 is a screenshot of the Patient Information screen.

FIG. 28 is a screenshot summarizing diabetes services which the patient is currently eligible to receive, which can be flagged.

FIG. 29 is a graph of Completed Bone Density DXA Exams by Month during a test of the HPO system provided herein for bone density screening follow-up.

FIG. 30 is a block diagram depicting the responsiveness of patients to outreach intervention in a diabetes outreach study.

FIG. 31 is a block diagram showing the results of patient outreach for colorectal cancer (CRC) screening by colonoscopy.

### DETAILED DESCRIPTION

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

This disclosure provides a health promotion outreach (HPO) system for increasing access of patients to medical services performed by a medical facility. The term, “increasing access of patients to medical services” means reducing barriers to patient's receiving care, for example, by alerting them, outside of doctor visits, that certain types of care are recommended, and scheduling the medical services directly, for example, while in telephone contact with the patient, without requiring the patient to visit his/her medical provider first. A medical facility can be any organization or group of organizations that perform medical services, e.g., clinic, hospital, doctor's office, corporate health care organization, and others known to the art, whether permanent or temporary, stationary or mobile, or comprised of single or multiple locations. The system disclosed herein enhances delivery of recommended medical services to patients, which means that they receive more recommended medical services than they would otherwise receive. Medical services include without limitation laboratory tests, radiological studies, medical procedures, and visits with clinicians. The system is especially useful for enhancing delivery of medical services for purposes of disease prevention and chronic disease management. Some chronic diseases for which the system is useful are: diabetes mellitus, chronic heart failure, coronary artery disease, hypertension, obesity, chronic obstructive pulmonary disease, asthma, cystic fibrosis, inflammatory bowel disease, bipolar disorder, psychosis, depression, arthritis, epilepsy, chronic renal insufficiency, hepatitis, lupus, and stroke.

An outreach system is one that initiates contact with patients outside the medical facility, such as through written, electronic, telephonic or personal communication by a representative of the medical facility. The representative does not need to have medical training, i.e., does not need to be trained or experienced in administering medical services to patients. In an embodiment hereof, the representative of the medical facility is an employee or member of an organization other than the medical facility.

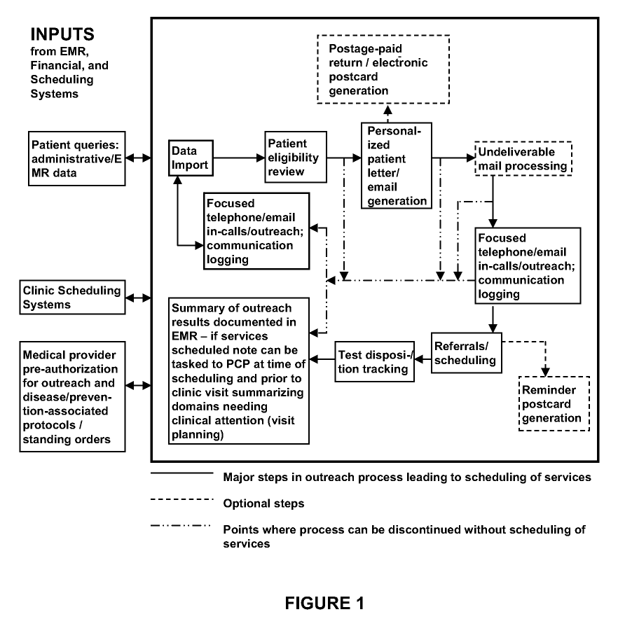

The system comprises a computer processor, which can be a single processor such as a desktop personal computer (PC) or other processor, two or more linked processors, such as processors connected to a central server, or any other configuration of electronic processing equipment known to the art. In an embodiment, e.g., as shown in FIG. 1, information in patient electronic medical records, which can be stored on separate or linked processors (indicated in the Figure as “Inputs,” is accessed by computer processors connected to a central server. All the linked processors shown in FIG. 1 can be referred to as a “processor.”

An electronic patient registry is an electronic database stored in the processor, in which patient records are kept, such as name, birth date, address, phone number, clinicians involved in their care, medical history elements, age, race/ethnicity, address, gender (sex), type of insurance, insurer, medical conditions, e.g., chronic disease diagnosis, and other patient information known to the art.

The processor also comprises electronic means operationally linked to the patient registry for accessing selected data from patient medical and administrative records of the medical facility and populating the patient registry with the selected data. Electronic means for populating the patient registry comprise processor elements known to the art in combination with algorithms providing instructions for extracting desired data elements from other databases and recording them in an appropriate field in the patient registry. Medical records can include information about a patient's medical history; time, place, date and outcome of medical services rendered to the patient; test results; biometric data (e.g. blood pressure, height, weight); medications; allergies; names and types of clinicians who have been involved in a patient's care; health insurance coverage; and patient demographic information (age, race/ethnicity, occupation, home and work addresses and phone numbers). Administrative records generally include patient contact information, billing and payment information, and medical diagnoses.

The processor is programmed with a tracking algorithm operationally linked with the patient registry. By “operationally linked” is meant that the algorithm comprises instructions for accessing selected information from the patient registry and using that information to perform operations such as recording the information in another location, sorting and displaying the data, and compiling the information with other information from the patient registry or elsewhere, and initiating further algorithms.

The tracking algorithm also comprises instructions for accessing, recording, sorting and displaying information about a patient's status in an HPO process and using that information to initiate further algorithms for performing additional operations, as described herein, well as other operations known to the art. The HPO process comprises at least the following steps: identifying and confirming eligibility of patients of the medical facility for selected medical services; establishing contact with patients who are confirmed to be eligible for the selected medical services, these patients generally being physically located outside the medical facility; scheduling the selected medical services for the patients; and recording the results of the selected medical services. The results can be recorded in the patient registry or in another database electronically operationally linked with the tracking algorithm. Determining a patient's current position in the process means identifying the steps of the process that have been performed and optionally identifying one or more further steps that need to be performed.

Determining patient eligibility for selected medical services means determining whether or not the patient meets criteria set by the medical facility, medical insurer, or medical provider. The criteria can include guidelines for medical care disseminated by professional organizations based on evidence or expert panel recommendations. Such guidelines are disseminated by organizations such as the American Cancer Society, the American Diabetes Association, U.S. Preventive Services Task Force, and the like. The eligibility criteria can also include requirements set by particular medical providers or medical facilities. As used herein, the term “medical provider” means any person who renders medical services, e.g. doctor, physician's assistant, nurse practitioner, laboratory technician, radiology technician, or other such person known to the art. The selected medical services can be selected from any and all medical services known to the art, including but not limited to chronic disease maintenance and preventive screening services, such as those pertaining to diabetes (e.g. laboratories, physician evaluations, eye exams), chronic lung disease (e.g. laboratories, spirometry, physician evaluations, rehabilitation services), screening tests for cancer (e.g. breast, cervix, colorectal, and prostate), cardiovascular disease and risk factor assessment (e.g. cholesterol, sphygmomanometry, electrocardiography), osteoporosis (e.g. bone densitometry), immunizations (e.g. influenza, tetanus, and pneumococcal vaccinations), and any combinations of such services. The system is especially useful for rendering services that accord with expert- and evidence-based guidelines for quality of care, but can be adapted for use with any type of medical service by one skilled in the art without undue experimentation.

The HPO system can also comprise electronic means operationally linked to the tracking algorithm for tracking confirmation of eligibility of patients for selected medical services by recording authorization from medical providers and/or insurance payers to perform the medical services. “Electronic means operationally linked to the tracking algorithm” for performing any function of the process include processor elements known to the art for retrieving, storing, sorting, compiling and displaying data, and also include databases stored in the processor or other processors, means for communicating with other processors, and means for inputting data into the processor.

Confirming eligibility for services involves one or more of the following activities: electronically or manually querying electronic and/or administrative records of the medical facility, to determine such information as that the patient has not already received the service within a stipulated interval, that there are no medical contraindications to the patient's receiving the service, that the patient is available to receive service, e.g., that the patient is still a patient of the medical facility, that the patient has provided consent to be contacted or that the patient has not previously indicated he/she does not wish to be contacted, that the medical provider has authorized the service, and that the insurance payer has authorized the service. In some cases, a medical provider or medical facility has given blanket authorizations for performing medical services, such as routine screenings or chronic disease management services for patients meeting preset criteria at predetermined intervals, e.g., set by medical guidelines as described above.

The HPO system can also comprise electronic means operationally linked to the tracking algorithm for generating written communications with patients confirmed as eligible for the selected medical services asking them to contact a representative of the medical facility, such as an employee of the HPO outreach system, for scheduling of the selected medical services. Means for generating written communications can include a mail-merge program known to the art that generates letters, post cards and/or email addressed to eligible patients.

The system can also comprise electronic means operationally linked to the tracking algorithm for inputting, recording and displaying results of the written communications to or from the patient (e.g. undeliverable mail, patient preferences conveyed by postage-return postcard). Such means include components known to the art such as keyboards, electronic storage media such as hard discs, and electronic display components such as computer screens.

The HPO system can also comprise electronic means operationally linked to the tracking algorithm for identifying and displaying contact information for patients requiring telephone contact. For example, a result of a written communication with the patients can be a communication back from the patient asking that a representative of the medical facility telephone the patient to schedule a medical service; or a result can be that after a specified period, e.g., two weeks, the patient has not responded to the written communication and should be telephoned to follow up.

The HPO system can also comprise means for prompting and recording results of telephone contact with the patient, which can be the same or similar to the means for recording results of written contact. Telephone contact can be prompted when the patient fails to respond to written communications after a specified period of time, e.g., two weeks, or if the patient has previously indicated he/she does not wish to receive written communications. Examples of typical results of telephone contact include: patient accepts services, patient has questions requiring follow-up communication, patient could not be reached, patient did not arrive for appointment, or services could not be completed.

The HPO system can also comprise electronic means operationally linked to the tracking algorithm for scheduling appointments for medical services for eligible patients and recording and displaying information about the scheduled appointments. These electronic means can include communication links and associated algorithms for automatically scheduling services with service providers such as laboratories and clinic personnel; or can simply comprise electronic means for recording details of manual scheduling performed by a representative of the medical facility via telephone or other communication means. Scheduling comprises giving the patient a time, place and date for performance of the services. The tracking algorithm also provides instructions for recording the scheduling details in the processor, and can also include instructions for directly recording scheduling details in the scheduling system of the medical service provider, or for activating scheduling software of the medical service provider.

The HPO system can also comprise electronic means operationally linked to the tracking algorithm for generating written reminders to patients of scheduled appointments. The algorithm includes instructions for accessing scheduling details and producing written reminders, as set forth above with respect to the initial written communications with patients. The reminders are generated from data input into the processor accessible to the tracking algorithm or can be generated by separate scheduling software known to the art. The reminders can include information as to the date, time, and location for delivery of the medical services as well as any special instructions for the patient.

The HPO system can further comprise electronic means operationally linked to the tracking algorithm for updating the patient registry. For example, results of written communications can include changes in contact information, information that the patient is no longer a patient of the medical facility, or other changes or additions to patient information. When such results of written communication with patients are input into the processor, the algorithm activates recording means to record it in the appropriate field(s) in the patient registry.

The HPO system can also include electronic means operationally linked to the tracking algorithm for recording the results of the outreach process and whether medical services were accepted by the patient, referred for authorization, scheduled, and completed. Such results can also include test results and other results known to the art and generally recorded in a patient's medical records.

The HPO system can also comprise electronic means operationally linked to the tracking algorithm for generating reports utilizing data stored in the processor. Examples of desired reports are compilations of data sorted by medical service provided, eligibility for service, medical provider, medical facility, test results, patient appointment, patient compliance, number and type of patient contacts, service dates, and/or patient demographics. Reports that document the process and final results of outreach for individual patients can also be generated in a format that allows them to be included in the medical record.

The HPO system described above is used to conduct a computer-implemented Health Promotion Outreach (HPO) process for increasing patient access to, and enhancing delivery of, medical services performed by a medical facility. The term “computer-implemented” means that a computer processor is used to implement steps of the process. The term “computer-assisted” can also be used to describe computer-implemented steps hereof, in which an operator, such as an outreach coordinator, inputs choices into the processor to initiate various algorithms and computer-implemented activities.

The HPO system, using its tracking algorithm, tracks the position of patients within the process. This HPO process comprises at least the following computer-implemented steps:
identifying and confirming eligibility of patients of the medical facility for selected medical services;
contacting patients who are confirmed to be eligible for the selected medical services, these patients being physically located outside the medical facility; e.g., at home or at work;
scheduling the selected medical services for the patients; and
recording the results of the selected medical services.
   The HPO process does not invariably require patients to personally see their medical providers advance for authorization of scheduling and performing the medical services.

The HPO process can also comprise obtaining and recording authorizations from medical providers to perform the medical services, and electronically storing a record of these authorizations in the system processor so that they are accessible to the tracking algorithm described above. This step can include manual operations (e.g., telephone contact with the medical provider) or can be performed mostly using electronic means (e.g., by email; by adding a patient's name to a database of patients requiring authorization of the medical services and receiving and storing an authorization received from the medical provider; or electronically comparing patient information with a list of patients preauthorized to receive the medical services.) performed manually or electronically.

The HPO process can further comprise obtaining and recording authorizations from insurance payers, medical providers and/or medical facilities to perform the selected medical services for the patients, and electronically storing a record of these authorizations in the system processor such that they are accessible to the tracking algorithm.

The HPO process can also comprise generating written communications with patients confirmed as eligible for the selected medical services asking the patients to contact a representative of the HPO system for scheduling of the selected medical services, transmitting the written communications to the patients, and electronically storing a record of the generated written communications in the processor such that they are accessible to said tracking algorithm. Generation of the written communications and scheduling can be done manually or electronically.

The HPO process can also comprise electronically storing a record of results of the written communications with the patients in the HPO system processor such that they are accessible to the tracking algorithm, and can further comprise causing the processor to retrieve and display contact information for patients requiring telephone contact.

The HPO process can also comprise making telephone contact with the patients requiring telephone contact and electronically storing a record of the telephone contacts in the system processor such that they are accessible to the tracking algorithm.

The HPO process of claim can also comprise scheduling appointments for medical services for eligible patients, which can be done manually or electronically, and electronically storing a record of the appointments in the system processor such that they are accessible to the tracking algorithm.

The HPO process can also comprise causing the system processor to electronically generate written reminders to patients of the scheduled appointments, transmitting the written reminders to the patients, and electronically storing a record of the generated written reminders in the processor such that they are accessible to the tracking algorithm.

The HPO process can also comprise recording the results of medical services performed for patients in the system processor such that they are accessible to the tracking algorithm.

The HPO process can also comprise generating reports utilizing data stored in the system processor.

The HPO process is typically used to reach out to patients to increase their access to guideline-concordant medical services. Guideline-concordant services are those prescribed by guidelines set by medical providers, facilities, or private or government health organizations, as discussed above.

The HPO system can be programmed to track and deliver any medical services (i.e., the same system handles multiple medical services); and to provide outreach and tracking for any combination of, or all, medical services for which an individual patient is eligible (e.g., a single written communication can be used to summarize any combination of, or all, medical services for which patient is eligible, and the tracking system can keep track of scheduling, authorization, and outcome for each of these services separately.)

The system allows tracking and enhances patient access to medical services selected including services pertinent to a chronic diseases, as known to the art and preventive services as known to the art, for example, immunizations, behavioral risk assessments, and screening tests for cancer, endocrine, pulmonary, gastrointestinal, psychiatric, developmental, musculoskeletal, neurological, genitourinary, and cardiovascular disease.

In addition to the exemplary aspects and embodiments described above, further aspects and embodiments will become apparent by reference to the drawings and by study of the following descriptions.

### Overview of the Health Promotion Outreach System (HPOS)

The purpose of HPOS is to increase access to and enhance the delivery of medical care by communicating with patients outside of clinical settings. HPOS enables outreach to patients to facilitate receipt of services reflective of health care quality as defined by organizations such as the National Committee for Quality Assurance (NCQA), the American Diabetes Association, and the United States Preventive Services Task Force.

### Core Characteristics and Objectives:

HPOS is based within and staffed by employees of a health care organization in which an ambulatory population of patients receive their care. Alternatively, HPOS can be provided as a third-party service to a health care organization that cares for an ambulatory patient population whenever said health care organization provides access to its information systems (e.g. scheduling and electronic medical records) by means of a formal business or other type of contractual arrangement. HPOS helps to realize a patient-centered medical home model by making use of up-to-date clinical information, streamlining access to clinical services, and facilitating timely communication between providers and patients.

HPOS uses electronic queries of administrative and/or electronic medical record data to identify patients eligible for medical services.

HPOS employs written followed by telephonic patient outreach in order to maximize the number of patients who are notified, educated about, and subsequently receive medical care. Outreach via letter, e-mail, and phone educates patients about their medical conditions and provides individualized recommendations for primary prevention and/or chronic disease management, thus supplementing the information that patients may or may not receive during face-to-face clinician visits. After receiving written correspondence, patients are invited to contact a call center to arrange for recommended services. If patients do not make in-calls after a specified period of time, and if they have not opted out of the service, coordinators can make out-calls to patients, thereby reaching those who are interested but have forgotten or not “gotten around” to calling.

While patients are on the phone, outreach coordinators schedule provider visits as needed but, whenever possible, also directly schedule laboratory tests, radiological studies, and subspecialty consultations. Such one-step scheduling maximizes patient convenience and adherence and increases the chance of successfully completing the recommended actions.

Providers agree prior to the implementation of particular services that this outreach protocol is medically appropriate. They authorize services through clinic protocols and/or by means of confirmatory electronic or written signatures on individual patient referrals and requisitions. The results of all tests and services are then communicated directly to authorizing providers so that they can provide appropriate follow-up. This workflow avoids concerns regarding provider autonomy and inappropriate HPOS interventions.

The results of patient outreach, including information obtained by telephone about patients' clinical status, concerns, and preferences, can be documented in the medical record for clinical continuity.

To carry out these tasks, registered nurse (RN)-level staffing has not been found to be necessary.

HPOS is an adjunctive and supportive system. In face-to-face encounters, providers care for patients as they always have. HPOS imposes minimal additional burden on medical providers and does not supersede established clinic routines. In contrast, HPOS can improve the quality and efficiency of in-person care by, for example, helping patients to complete laboratory testing several days before clinic appointments.

As a “clinical extender,” the HPOS helps medical providers seamlessly identify, reach out to, communicate with, and provide higher quality care to their patients. Because the HPOS is able to carry out routine tasks that do not require the direct involvement of physicians, more time is made available for medical providers to focus on the complexities of patient care during actual face-to-face encounters. In addition, patient access to providers is improved when clinic slots are used more judiciously.

HPOS can change dynamically: elements of primary prevention and chronic disease outreach can be added, simplified, enhanced, or eliminated depending on capacity constraints, data resources, the extent to which HPOS goals are being met, and the priorities of payers, patients, and medical groups. As depicted in Table 3, HPOS and each of its services can also vary in terms of underlying data queries; mechanisms and content of patient outreach; protocols for verifying eligibility and approving and scheduling services; staffing requirements; and number and type of ambulatory clinics supported. For example, administrative data queries (e.g. billing claims) that identify when services were last provided can promote future receipt of services at guideline-based intervals, such as physician visits and hemoglobin A1c evaluations every six months for patients with diabetes. Electronic Medical Record (EMR) queries, on the other hand, can also facilitate provider visits when diabetic patients are not meeting guidelines for blood pressure control and the use of aspirin and statins.

TABLE 1
HPOS characteristics and scalability

| HPOS | | |
|---|---|---|
| characteristic/ | Intensity/sophistication/HIT infrastructure | |
| function | LESSER | GREATER |
| Based within or | Primary care locus | Stand-alone unit serving |
| contracted by a full- | | primary care and |
| service health care | | subspecialty ambulatory |
| organization | | practices |
| Electronic data | Administrative data | EMR data (medical problem |
| queries identify | (demographic, billing | lists, medications, vital signs, |
| patients eligible for | claims, scheduling records) | biometrics, laboratory/test |
| guideline-concordant | High-risk/high cost patients | results) |
| services | Selected primary prevention | Population management (all |
| | or chronic disease services | patients with a disease |
| | Promote testing/evaluations | condition) |
| | at recommended intervals | Comprehensive primary |
| | | prevention and chronic |
| | | disease services |
| | | Promote attainment of |
| | | behavioral, laboratory, and |
| | | clinical goals |
| Patient outreach in | Simple written reminder that | Written reminder mentioning |
| written form followed | service is due | name of provider and most |
| by telephonic contact | Outreach focused on a set | recent clinical results, and |
| through a call center | of services for a single | including educational |
| | primary prevention or | materials |
| | chronic disease condition | Outreach focused on |
| | Written outreach by postal | providing services for all |
| | service | HPOS primary prevention |
| | Patient in-calls only | and chronic disease |
| | | conditions for which patient |
| | | is eligible |
| | | Written outreach by e-mail ± |
| | | postal service |
| | | In-calls by and out-calls to |
| | | patients |
| Streamlined | Coordinator telephonically | Coordinator schedules |
| scheduling of | transfers patients to | services directly |
| services | relevant scheduling units | Written reminders regarding |
| | No written reminders | dates and locations of |
| | regarding dates and | scheduled services |
| | locations of scheduled | |
| | services | |
| Providers authorize | Standing orders allow | Providers verify individual |
| all orders | coordinators to verify patient | patient eligibility before |
| | eligibility; providers | outreach and authorize |
| | authorize orders after | orders after patient |
| | patient acceptance | acceptance |
| Providers receive all | Paper | E-mail or EMR tasks |
| reports and test | | |
| results and are | | |
| responsible for | | |
| clinical follow-up | | |
| Staffed by outreach | Non-RN personnel | RN personnel supervising a |
| coordinators | | team of non-clinical |
| | | personnel for stratified |
| | | interventions. RN also |
| | | attends to needs of patients |
| | | with higher risks/costs |

While HPOS coordinators can verify patient eligibility for many basic services, other services—especially ones that are somewhat more costly, time-consuming, or risky (e.g. screening colonoscopy)—are likely to require prompted medical provider review for individual patients.

HPOS can focus on patients who are likely to develop complications and incur high costs, but it can also support true population management by tracking clinical parameters and providing outreach for all patients with a particular disease condition (an arrangement that yields better pay-for-performance measures and more complete and up-to-date registries).

In general, HPOS targets patients who have completed at least one clinic visit in a healthcare organization (and who as a result have at least one record in an administrative or EMR database). However, if a health plan furnishes the names of its enrollees who have been assigned to a primary care provider (PCP) within the healthcare organization, HPOS can also facilitate introductory clinic visits for patients who have not yet completed one.

The same HPOS infrastructure that facilitates care for patients with chronic illness can promote primary prevention (e.g. cancer screening and immunizations) for a general clinic population. In doing so, less intensive outreach is appropriate for some services compared with others. For example, patients due for a yearly mammogram might receive a simple reminder letter with advice to call and schedule the test; meanwhile, patients with congestive heart failure might receive detailed written summaries of their recent laboratory values, tailored educational materials, multiple recommendations for care, and one or more phone calls from an HPOS coordinator.

To maximize patient convenience and adherence, patients should not be required to make multiple phone calls. Thus, if a particular service is definitely (or extremely likely to be) covered under a particular health plan, the service should be scheduled on the first occasion that telephonic contact is established. Ideally, an HPOS outreach coordinator will be able to do this directly through a computerized scheduling system. When not feasible, however, interested patients can be telephonically transferred from an HPOS call center to an appropriate scheduling unit. At University of Colorado Hospital (UCH), for example, HPOS coordinators schedule DXA exams directly through the radiology scheduling system. On the other hand, diabetic patients needing yearly retinal exams are transferred to ophthalmology department schedulers because complex algorithms are used to determine appointment dates and the availability of particular providers. In some cases, it will not be possible to schedule a service immediately—perhaps because insurance pre-authorization is required. The HPOS coordinator can nonetheless streamline future scheduling by recording patient preferences related to call-back times, contact phone numbers, and appointment dates.

### HPOS Information Management System

An HPOS information management system has been developed to support a novel business method. A version of this system is depicted in FIG. 1. The column on the left side labeled “Inputs” represents information from sources that can be by an operator of the method herein. The information can be stored on separate sources such as individual or multiple computer processors. The larger box to the right represents a single processor, or multiple linked processors, for example all connected to a single server, programmed to perform the method steps described herein. In embodiments, each box can represent a method step as labeled, and can be associated with a computer processor with a screen display or series of displays which give the operator options for proceeding with the method.

Patients who appear to be eligible for particular services are identified through administrative or electronic medical records (EMR) data queries that extract scheduling information, patient contact information, patient demographic information and the like as well as clinical data about the patient's health. This information is then cross-referenced against disease and/or prevention registries and imported on a real-time, daily, weekly, or monthly basis into the information management system. Outreach coordinators then review this information to confirm individual patient eligibility for offering medical services through outreach. The eligibility review step can be computer assisted via a processor programmed with an algorithm to weigh eligibility criteria or can be carried out through manual review of the EMR. As part of the eligibility review process, the outreach coordinator can also obtain pre-authorization for individual patient outreach from medical providers. If the patient is ineligible for services, as shown by the dashed and dotted line in FIG. 1, the patient registry is updated with this information and a summary of outreach results can be placed in the EMR records for the patient.

If the patient is eligible for the services, personalized communications such as letters and/or emails to patients mentioning the clinician's approval and involvement, are then generated that include specific recommendations for care, encouragement to telephone the HPOS call center and, when pertinent, individualized summaries of clinical indices (e.g. dates of last appointment with a provider, laboratory and biometric values). The communications to the patients can include services for which the patient is eligible, specific invitations to contact the coordinator, and educational information. Optionally, communications are accompanied by a postage-paid return postcard, or electronic response invitation in the case of email communications, that allows patients to update their contact information and specify whether they are no longer receiving care within the health system or from identified providers, are uninterested in scheduling the service(s) offered, wish to proceed with scheduling the service(s) given particular time and date preferences, wish to opt out of HPOS altogether, or have completed the service(s) in another setting. If patients do not respond to the communications or make in-calls to the outreach coordinator within a specified period of time, HPOS coordinators commonly (although not invariably) initiate outcalls. All telephonic communication is logged, and patient records are closed whenever a predetermined number of call attempts or days have been reached. If the postcard (electronic or regular mail) is returned as undeliverable, or if the patient responds to the communication from the coordinator declining services, as indicated by the dashed and dotted line in FIG. 1, the patient registry and EMR records are updated as discussed above.

If patients agree to proceed with recommended service(s) by responding to the initial communication, or in subsequent telephone or email communication from the outreach coordinator, the outreach coordinator generates referrals and insurance pre-authorization requests as needed and facilitates the scheduling of appointments. These details are logged and tracked within the information management system in order to facilitate the completion of steps from insurance pre-authorization to scheduling. Once services have been approved and scheduled, reminders, such as postcards or emails can be generated by the system and sent to patients reminding them about the date, time, and location of the service(s) as well as information about canceling and rescheduling.

A separate module allows for logging final outcomes of the scheduled service(s), including adherence and test results, review by PCP, and other tracking information, thus making it possible to generate summary HPOS reports at the level of individual medical providers, clinics, and types of service (e.g. number and proportion of HPOS patients diagnosed with osteoporosis). These reports can then be used to inform clinical and financial analyses. From data importation to the final disposition of scheduled service(s), pertinent information obtained from patients is used to update and maintain registries, administrative records, clinical EMRs, and scheduling systems. In addition, written summaries of all completed HPOS steps can be generated at any point and documented in the EMRs.

### HPOS has Advantages Over Traditional Preventive Care Delivery Mechanisms

For an average-sized panel of 2,500 patients with an age and sex distribution similar to the US population, a PCP would require 7.4 hours per working day in order to provide all the primary preventive services recommended by the United States Preventive Service Task Force (USPSTF) (Yarnall et al., 2003). Clearly, this is untenable, and all the more so because there is evidence that increasing amounts of specialty care are shifting to primary care settings, further increasing the pressure on PCPs (St Peter, Reed, Kemper, & Blumenthal, 1999). In fact, in the vast majority of instances, identifying and referring patients for primary prevention services does not require physician-level expertise. Thus HPOS can take over many of the routine functions of identifying eligible patients, notifying them about recommendations for preventive care, and helping them to receive these services often without the intermediate step of seeing medical providers ahead of time for referrals. On the other hand, HPOS can also schedule patients to see their medical providers at appropriate intervals in order to complete general health assessments. In this regard, an important category of patients includes those who are assigned to, but have never seen, a PCP within a healthcare organization. If insurance companies or health plans are able to furnish lists of patients assigned to a medical group, this data can be imported into an HPOS information management system in order to facilitate outreach and introductory primary care visits. At the same time, the HPOS can mail health assessment forms (e.g. smoking, family, and personal medical histories, medication lists, and allergies) to be completed by patients prior to their appointments.

### HPOS has Advantages Over Chronic Disease Management Programs (DMPs)

Chronic disease management programs (DMPs) are usually carried out by third parties. (Although HMO-exclusive integrated health systems have their own internal disease management programs, these should be considered part of a Chronic Care Model as described in the next section.) DMPs are designed to minimize the development of serious complications among patients with chronic illnesses (Disease Management Association of America (DMAA)). DMPs promote adherence to clinical practice guidelines and strive to curtail the utilization of excessively costly and non-evidence-based services. DMP case managers, often registered nurses, follow a panel of high-risk/high-cost patients and contact them at regular intervals in order to provide education, self-management support, and encouragement to adhere to recommendations for laboratory and clinical evaluations. When feasible, DMPs also furnish performance reports to medical providers in order to promote improved quality of care. Although they are meant to serve important purposes, evidence is mixed about whether many types of DMPs save money and improve clinical outcomes (Fitzner et al., 2005).

HPOS has several advantages over third-party DMPs. First, because DMPs are managed by organizations external to and unaffiliated with the health systems in which patients receive their care, DMPs tend to have incomplete and delayed access to clinical information (e.g. laboratory values and clinic visit records), limited ability to facilitate timely patient access to recommended services, and inefficient and belated mechanisms for providing feedback to providers. In addition, whether warranted or not, patients, medical providers, and clinic administrators sometimes view DMPs as having a primary interest in cost containment rather than quality of care and patient satisfaction. HPOS, on the other hand, by virtue of its integration or close linkage with health care systems where patients receive their care, has complete and immediate access to clinical information and appointment scheduling as well as the ability to broker information rapidly and efficiently among providers and patients. HPOS can also capitalize on established therapeutic alliances because all HPOS outreach is carried out “on behalf of” medical providers and explicitly includes them in approving orders and reviewing results. Finally, while DMPs typically focus on a single chronic illness, HPOS is not only able to facilitate guideline-based care for multiple chronic conditions simultaneously, but can do the same for primary prevention services.

In principle, HPOS, like most DMPs, can employ RNs as outreach coordinators to promote patient self management (Dorr, Wilcox, McConnell, Burns, & Brunker, 2007). In our experience, however, HPOS coordinators do not necessarily have formal clinical training. Nonetheless, because the coordinators work within the health care system, they can facilitate patient receipt of guideline-based services more efficiently and economically than most DMP RN case managers. They can also help patients access educational and self-management programs that might be available within the health care organization and community.

### HPOS Complements and Supports the Chronic Care Model (CCM)

The Chronic Care Model (CCM) describes a system for providing high quality care to patients with chronic illnesses (Bodenheimer, Wagner, & Grumbach, 2002; Rothman & Wagner, 2003; Wagner, Austin, & Von Korff, 1996). A completely-realized example of the CCM utilizes substantial HIT infrastructure to provide decision support at the point of care as well as the ability to track and report clinical processes and outcomes. In many ways and in most venues, the CCM remains an idealized picture of what health care should become. It has been most fully elaborated within HMO-exclusive integrated health systems (Wallace, 2005), but there is evidence for significant chronic disease quality of care improvements even within small clinical practices that incorporate only a few elements of the CCM (Nutting et al., 2007; Ouwens, Wollersheim, Hermens, Hulscher, & Grol, 2005).

Certain features of HPOS are not generally emphasized in the CCM. For example, more overtly than the CCM, HPOS identifies population management as one of its key functions. Similarly, although CCM infrastructure can be used to support primary prevention (Glasgow, Orleans, & Wagner, 2001), this possibility is rarely made explicit in the CCM as it is in HPOS. For the most part, descriptions of the CCM have also not highlighted the value of patient outreach, assigning routine healthcare tasks to non-medical personnel, and promoting patient convenience by omitting antecedent provider visits whenever possible. In these ways, HPOS is properly viewed as a system that enhances and promotes the CCM.

Table 2 compares key features of DMPs, the CCM, and HPOS. In general, DMPs compete with or are duplicative of many CCM and HPOS functions. On the other hand, HPOS and the CCM are complementary and mutually supportive: HPOS facilitates guideline-based care through patient outreach while the CCM focuses on improving care through traditional face-to-face patient-PCP encounters; enhancements to HIT infrastructure make the activities of both HPOS and the CCM more robust; HPOS offers a powerful mechanism for improving information flow among providers and patients; and continuous HPOS outreach produces more accurate and up-to-date population registries (e.g. identifying patients who die, leave the healthcare system, move, and change phone numbers) than is possible with the CCM alone. Finally, HPOS supports CCM's focus on providing care that is patient-centered (Davis, Schoenbaum, & Audet, 2005): through outreach, and often in anticipation of provider visits, HPOS helps to clarify patient values, preference and needs; it provides additional channels for informing and educating patients; it streamlines access to care; and it results in better coordination of care.

TABLE 2
Key characteristics of three healthcare delivery methods*

| Characteristic | 3^{rd} party DMP | HPOS | CCM |
|---|---|---|---|
| Evidence- | Strong emphasis | Strong emphasis | Strong emphasis |
| based/expert | | | |
| guidelines | | | |
| Physical location, | External to locus | Integrated into | Integrated into |
| affiliation | of patient care, | specific clinical | specific clinical |
| | unaffiliated | practice(s) or as | (e.g. primary |
| | | stand-alone unit within | care) practice |
| | | larger health system | |
| Usual mode of | Telephonic with | Telephonic with | Face-to-face with |
| patient contact | RN case | outreach coordinator | medical provider |
| | manager on | on behalf of patient's | |
| | behalf of 3^{rd} party | medical provider | |
| Access to patient | Delayed, often | Rapid, complete | Rapid, complete |
| clinical information | incomplete | | |
| Ability to schedule | Poor | Good | Good |
| services and arrange | | | |
| follow-up &planned | | | |
| provider visits | | | |
| Feedback to medical | Delayed, | Rapid, efficient | Rapid, efficient |
| providers | inefficient | | |
| Decision support at | Poor | N/A (HPOS promotes | Explicit element of |
| point of care | | guideline-based care | CCM |
| | | by contacting patients | |
| | | outside of clinic | |
| | | setting) | |
| Responsibility for | RN case | Outreach coordinator | PCP plays direct |
| promoting patient | manager and | can facilitate patient | role ± ancillary |
| self-management | DMP-based | participation in clinic- | clinic |
| | educational | based programs | staff/programs |
| | programs | | |
| Cost containment | Strong emphasis | Can be tailored | Minor emphasis |
| Community alliances | Poor | Outreach coordinator | Explicit element of |
| (e.g. support groups, | | can facilitate patient | CCM |
| exercise programs, | | participation in | |
| senior centers, etc.) | | community services | |
| Ability to manage | Poor | Can be tailored | Good |
| multiple co- | | | |
| morbidities | | | |

DMP = Disease management program;
HPOS = Health Promotion Outreach System;
CCM = Chronic Care Model.
Best practice coded in grey.

Not only can HPOS be used to augment CCM in practices ready to support wholesale practice redesign, it can also be implemented as a first step toward systems-based practice. For instance, practices that generate administrative level data, but do not have an EMR, can take advantage of the care improvements that accrue from HPOS. An example of a primary prevention practice improvement, administrative data can be used to generating a report of patients who are female, older than age 65, and without a record of bone densitometry. This administrative data set can be imported into the HPOS information system and a non-medically trained assistant can begin generating customized letters, postcards and telephone calls to patients who appear to be candidates for bone density screening. Patients who agree to screening can be scheduled, and orders and results can be placed into the paper medical record for provider review, thereby improving screening rates without substantially affecting provider workflow or increasing time burden. As a practice gains sophistication, particularly with implementation of an EMR with coded problem and medication lists and laboratory data, even more opportunities for outreach become available. In this way, HPOS can precede and support a full implementation of the CCM.

FIG. 1 is a flow sheet showing the major steps in the HPOS described herein.

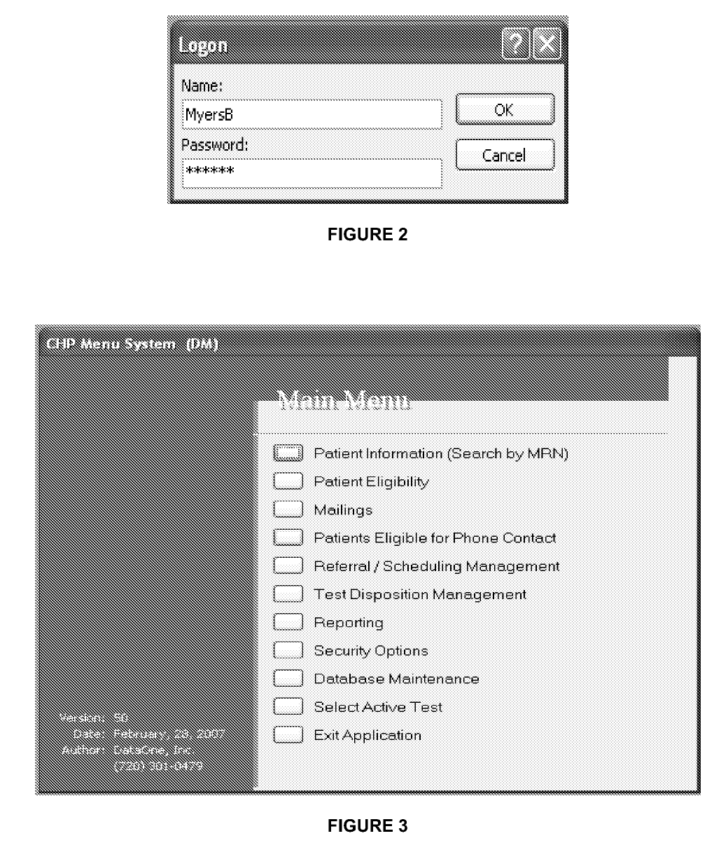

FIG. 2 is a screenshot of the Logon screen that appears upon opening of the software. In the embodiment shown herein, the software is a Microsoft Access application that can be accessed via a shortcut once set-up is complete. The software allows multiple users and is secured using individual logins and passwords.

FIG. 3 is a screenshot of the Main Menu. After the software is opened and a user enters their username and password they are automatically redirected to the Main Menu screen. The main menu allows a user to choose from options offered by the system, namely, Patient Information (which can be searched by Medical Record Number (MRN); Patient Eligibility; Mailings; Patients Eligible for Phone Contact; Referral/Scheduling Management; Test Disposition Management; Reporting; Security Options; Database Maintenance; Select Active Test; and Exit Application. Of note: the current medical service is always displayed in parenthesis at the top of the menu. In this case (DM) is listed indicating the active medical service is a diabetes mellitus test.

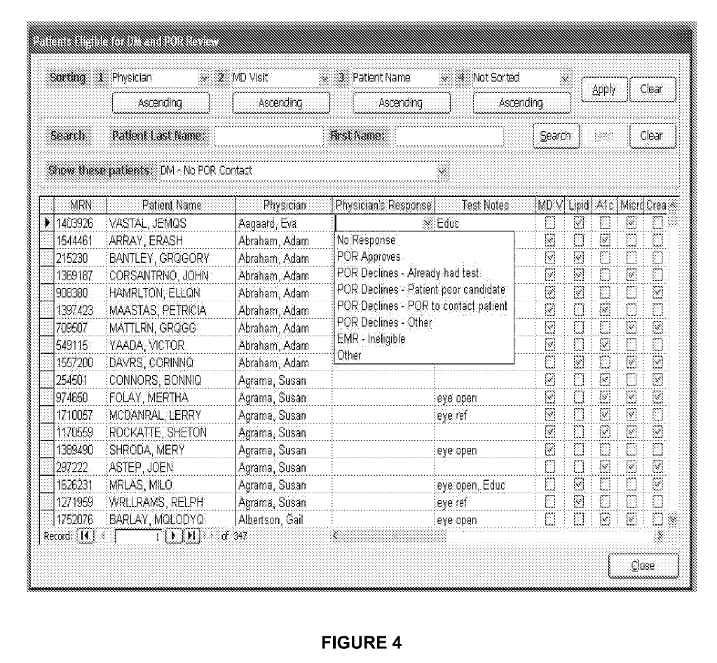

FIG. 4 is a screenshot of the Patient Eligibility screen, which can be selected on the screen shown in FIG. 3. The Patient Eligibility screen lists patients that have been imported (i.e. a process by which patient records in another database are used to populate a patient registry in the tracking system) for a specific test that requires individual medical review to confirm eligibility for outreach. As shown, the user can sort up to four fields or search by a patient's first or last name. The user can specify which group of patients they would like to view and then see their individualized information. This individualized patient information includes: medical record number, name, physician, physician's response, test notes, tests due, last service date, date the physician of record (POR) was contacted to provide information about patient eligibility, days since POR contact, insurance plan, age, date patient contacted and days since the patient contact. These columns of information can be arranged and sorted in any order based on the user preference and need. After the medical record review, the user can specify the Physician's Response as shown in the image below.

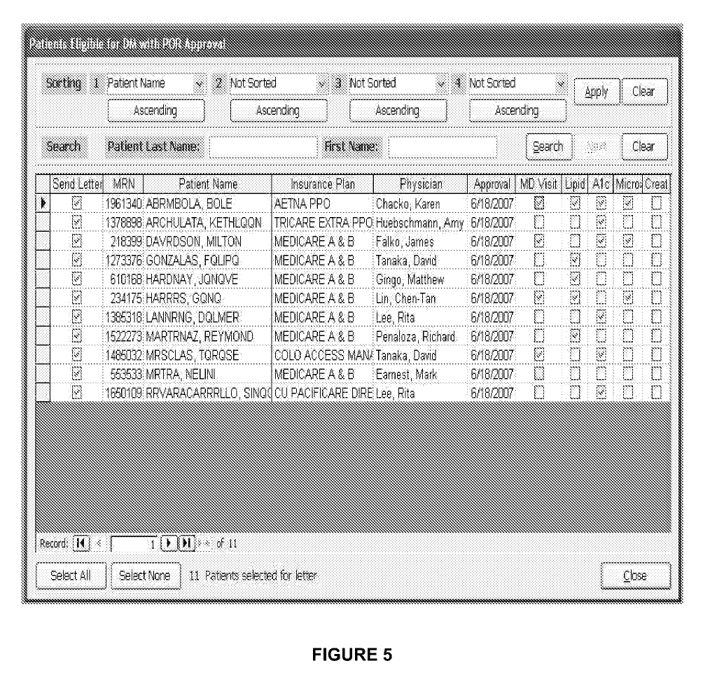

FIG. 5 is a screenshot of the Patient Eligibility screen, displaying only those patients who have received final confirmation of eligibility for outreach from the POR.

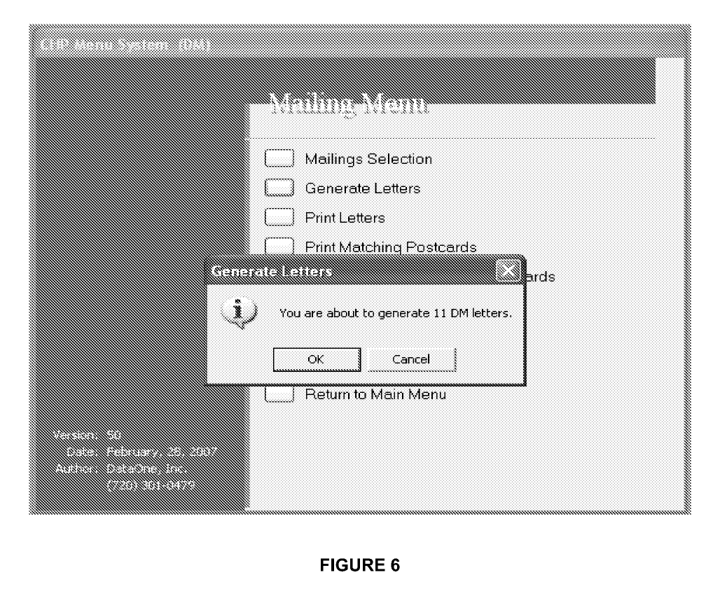

FIG. 6 is a screenshot of the Mailing Menu screen, which can be selected from the Main Menu screen shown in FIG. 3. Selecting “Mailing Selection” allows the user to select which of the eligible patients will be contacted via mail. Again, this screen allows multiple sorts and searching. Other options displayed on the mailing menu are Generate Letters; Print Letters; Print Matching Postcards; Postcards-Process received cards; Reprint a Letter; Reprint a Postcard; Select Active Test; and Return to Main Menu. Superimposed on FIG. 6 is a dialog box allowing okaying or cancelling of the command to print letters. “Print Matching Postcards” also allows cancelling or okaying the printing of matching postcards. Okaying the commands automatically generates or prints tailored letters and return postcards from the templates stored in the system for the type of medical service involved.

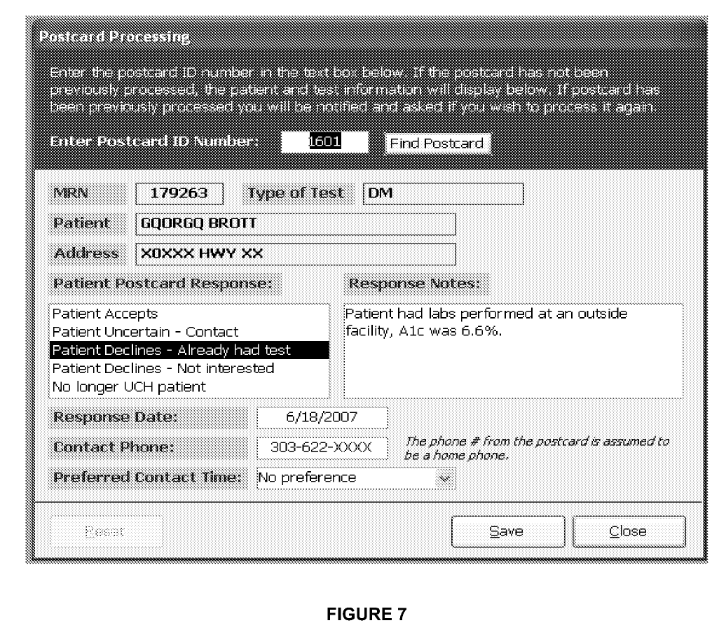

FIG. 7 is a screenshot of the Process Received Postcards screen which can be accessed by selecting “Postcards—Process received cards” on the Mailing Menu shown in FIG. 6. If a return postcard is sent back by the patient, the information on the card can be processed and entered into the system as shown on the screen. Each postcard is given a unique identification number. This screen has fields for entering that the patient accepts the service, the patient is uncertain and should be contacted, the patient declines the service because the service has already been performed, the patient declines the service because he/she is not interested, and the patient is no longer a patient of the medical facility, as well as a field for notes, response date, contact phone, and preferred contact time.

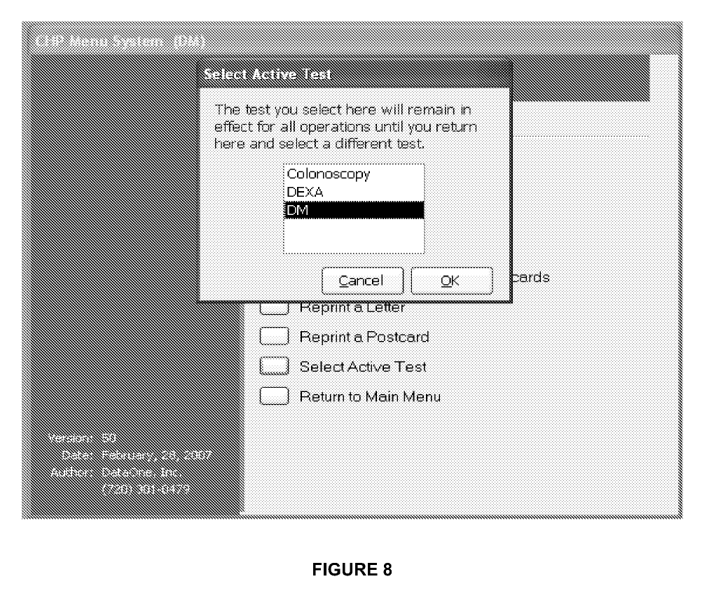

FIG. 8 is a screenshot of the Select Active Test screen, which can be accessed by selecting this screen on the Mailing Menu shown in FIG. 6 or the Main Menu shown in FIG. 3. This allows the user to process information and generate mailings for selected tests.

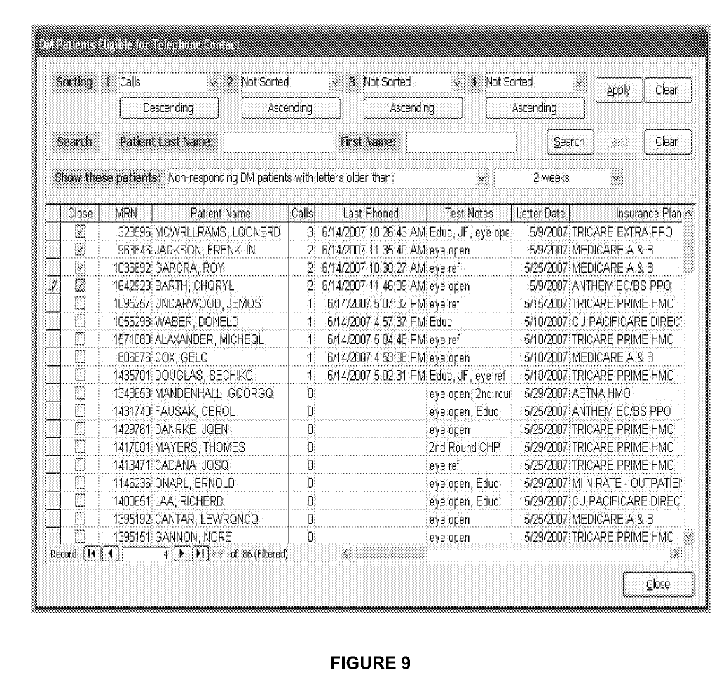

FIG. 9 is a screenshot of the Patients Eligible for Telephone Contact screen that can be accessed by selecting this screen on the Main Menu shown in FIG. 3. This screen allows users to see which patients are eligible for telephonic outreach. In this screen the following data is listed: Medical Record Number (MRN), patient name, number of calls, date of last phone call, test notes, letter date, insurance plan, birth date, physician, preferred contact time, tests due and checkboxes for postcard necessary and closing the test. As is possible on the Patient Eligibility screen, the user is able to sort, search and rearrange the columns as necessary. Another feature in this screen is the ability to list only patients who have not responded after a set period of time (all, 1 week, 2 weeks, etc). The user can also show only patients who have left a voicemail or who have requested contact via a postcard.

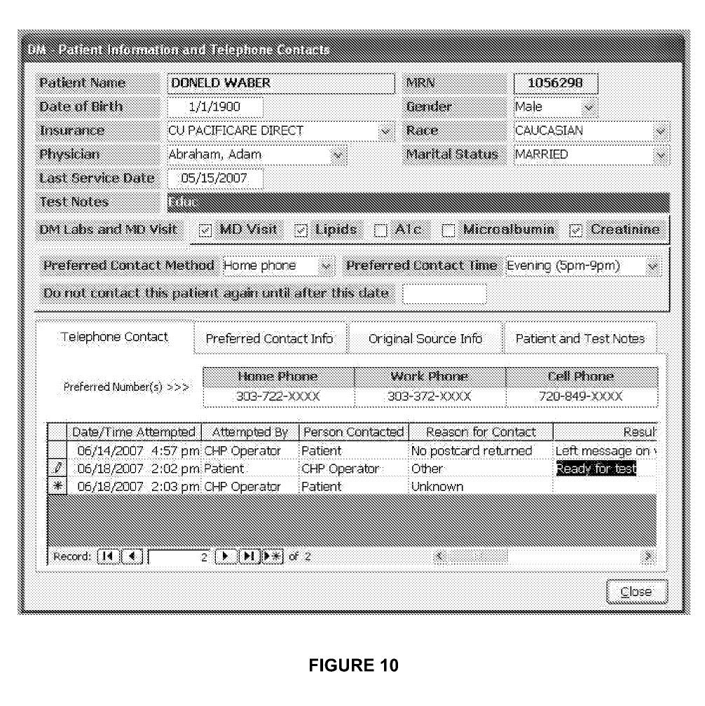

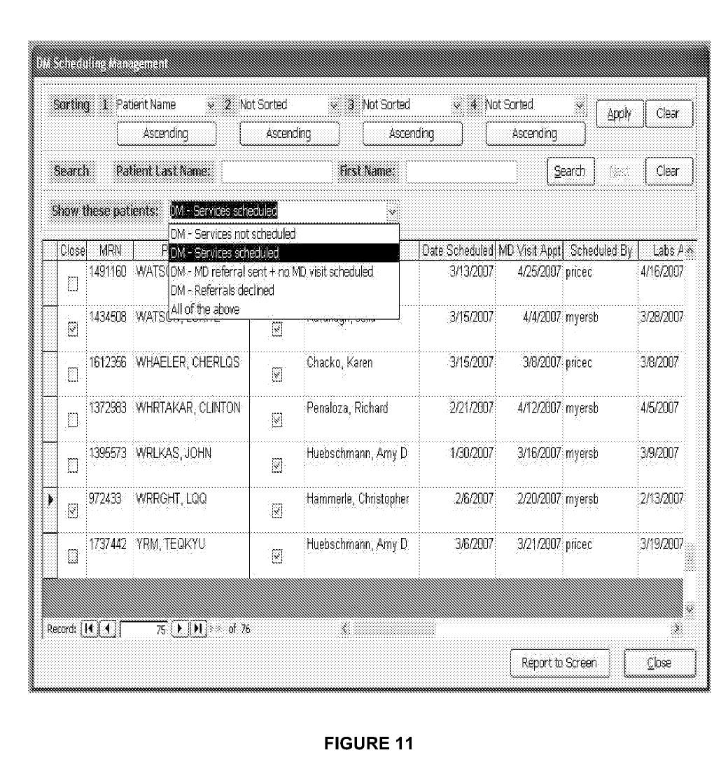

FIG. 10 is a screenshot of the Patient Information and Telephone Contacts screen, which can be accessed by double clicking the patient's name on the Patients Eligible for Telephone Contact screen shown in FIG. 11. Results of the telephone contact can be recorded on this screen, which contains fields for patient information, medical services due, contact information including preferences, and listing each call with results.

FIG. 11 is a screenshot of the Scheduling Management screen, which can be accessed by selecting “Referral/Scheduling Management” on the Main Menu screen shown in FIG. 3. This screen lists the patients who have accepted services and ready to be scheduled and/or have a referral entered. The information included on this screen that was not present on previous screens are the date scheduled, date of labs and appointments, and who scheduled the appointments.

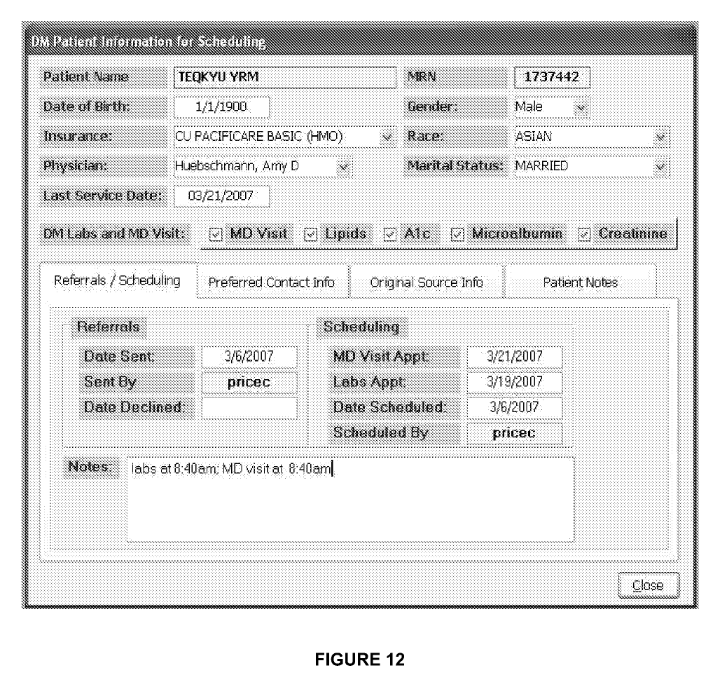

FIG. 12 is a screenshot of the Patient Information for Scheduling screen, which can be accessed by selecting a patient's name on the Scheduling Management screen shown in FIG. 11. This screen includes patient referral and scheduling information with notes.

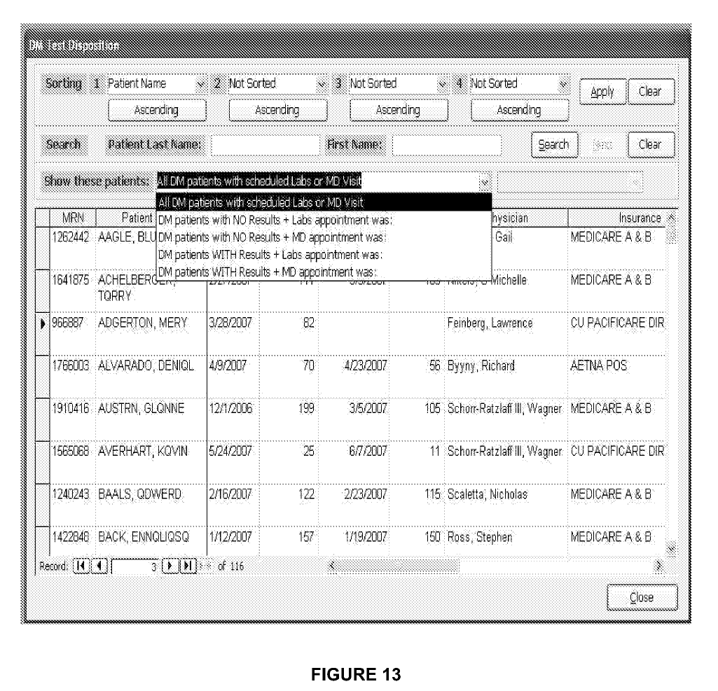

FIG. 13 is a screenshot of the Test Disposition screen, which can accessed by selection of “Test Disposition Management” on the Main Menu screen shown in FIG. 3. This screen lists all the patients who are scheduled for medical services (lab work, doctor visits, etc.). This list can be sorted by date, listing only patients with or without results from a specific time period, or by other fields.

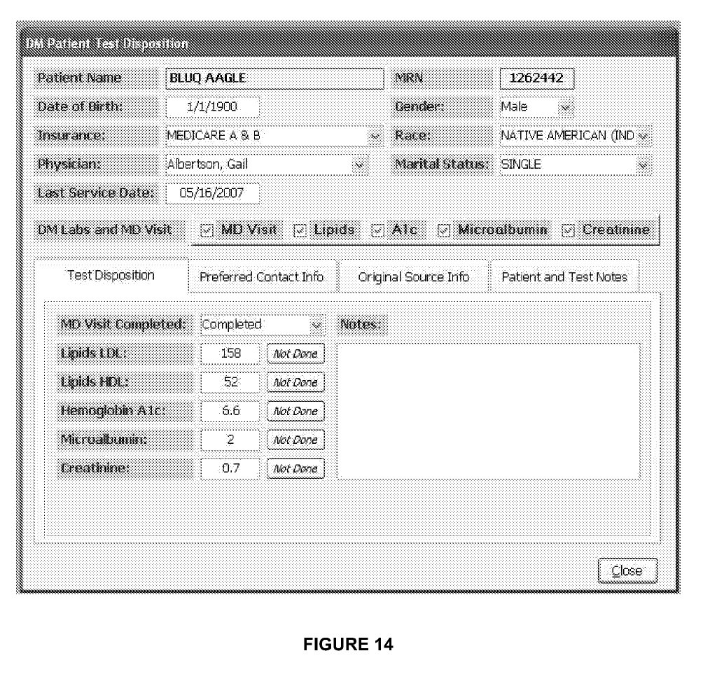

FIG. 14 is a screenshot of the Patient Test Disposition screen, which can be accessed by selecting a patient name from the Test Disposition screen shown in FIG. 13. Detailed results of medical services can be entered on this screen.

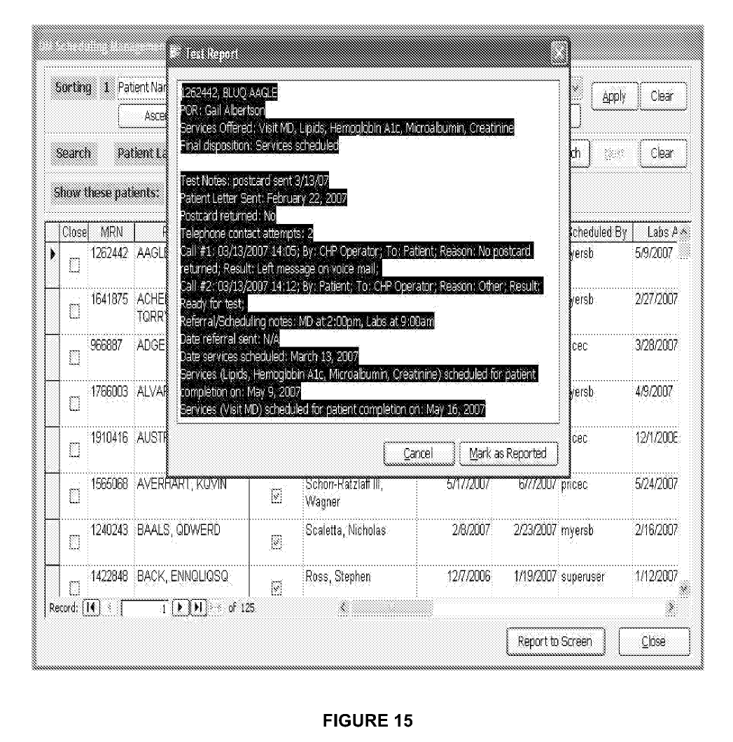

FIG. 15 is a screenshot of a Test Report screen, which can be accessed by selecting a patient's name and “Report to Screen” from the Scheduling Management screen shown in FIG. 11. Tracking information regarding the steps in the process that have been completed are displayed on this screen. These individual reports appear in text that can be copied and pasted into another field, such as the patient's electronic medical record with the medical facility.

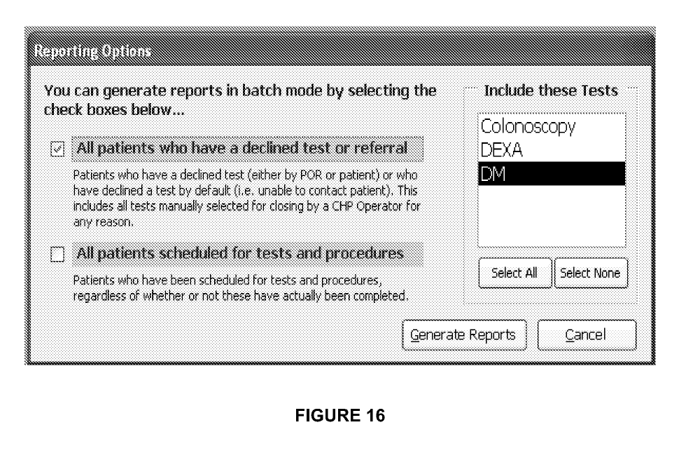

FIG. 16 is a screenshot of the Reporting Options screen, which can be accessed by selection of Reporting Options on the Main Menu screen shown in FIG. 3. This screen allows selection of batch reports, for example, for all patients who have declined a test or referral or all patients scheduled for tests or procedures, for a particular type of medical service.

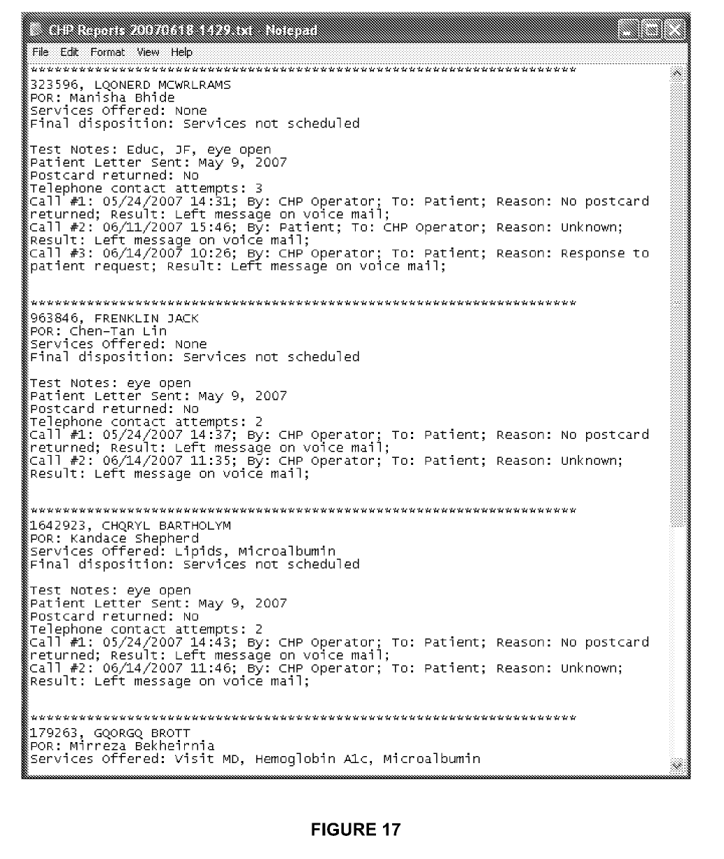

FIG. 17 is a screenshot of a text report generated by making selections for a batch report on the Reporting Options screen shown in FIG. 16.

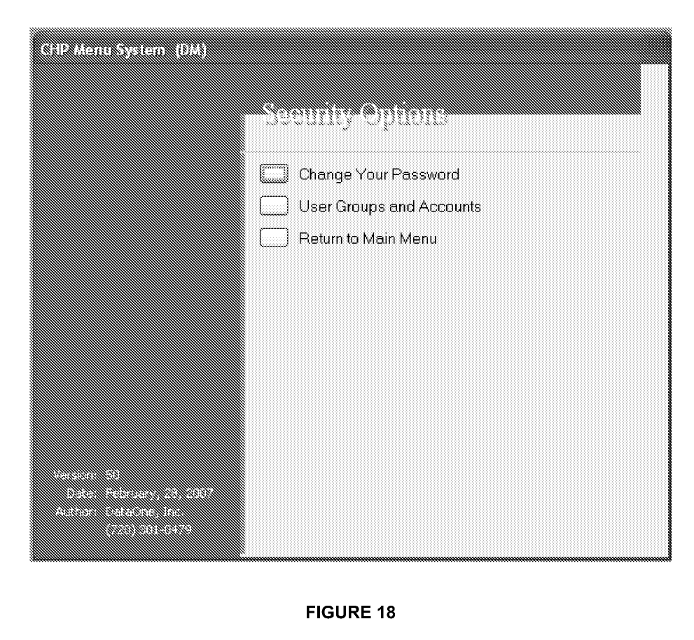

FIG. 18 is a screenshot of the Security Options menu, which can be accessed by selecting “Security Options” on the Main Menu shown in FIG. 3. This screen allows the user to change his or her password, to select user groups and accounts, or to return to the main menu.

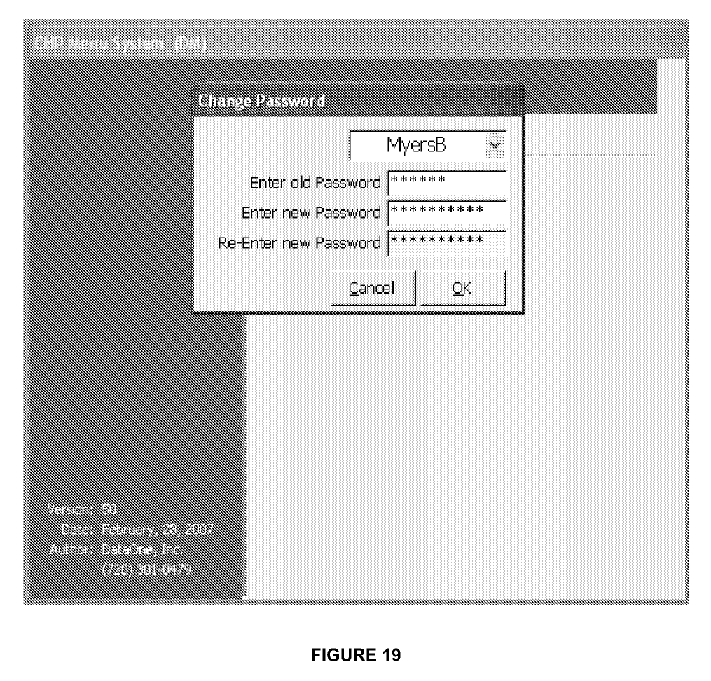

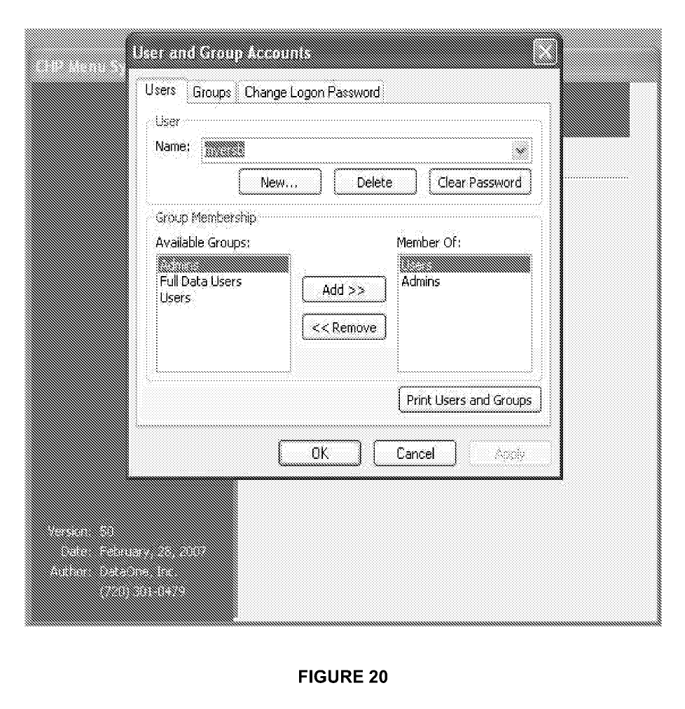

FIG. 19 is a screenshot of the Change Password screen accessed by selecting “Change Your Password” on the Security Options menu shown in FIG. 20. The user can change his or her password using this screen.

FIG. 20 is a screenshot of a User and Group Account screen, which can be accessed by selecting this option on the Security Options menu shown in FIG. 18. This screen allows the user to change his or her access to various levels of or subsets of information stored in the system. For example, selecting “Admins” from this screen allows the user to add, remove, and configure medical services and tracking processes in the application; selecting “Full Data Users” allows the user access to all patient data for all medical services currently active within the application, and selecting “Users” allows the user access to selected medical services only. The user's logon password can also be changed from this screen.

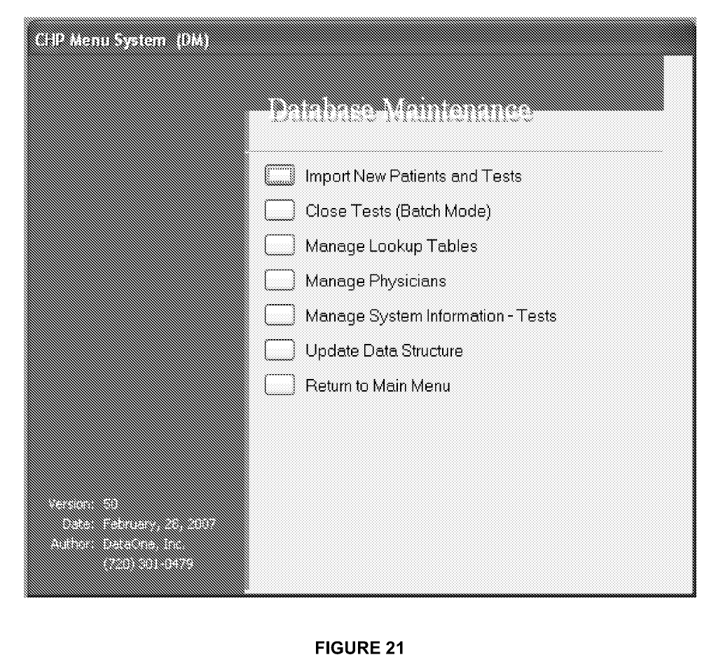

FIG. 21 is a screenshot of the Database Menu Management screen, which can be accessed by selecting this choice on the Main Menu screen shown in FIG. 3. This screen allows choices for Close Tests (Batch Mode), Manage Lookup Tables, Manage Physicians, Update Data Structure, and Return to Main Menu.

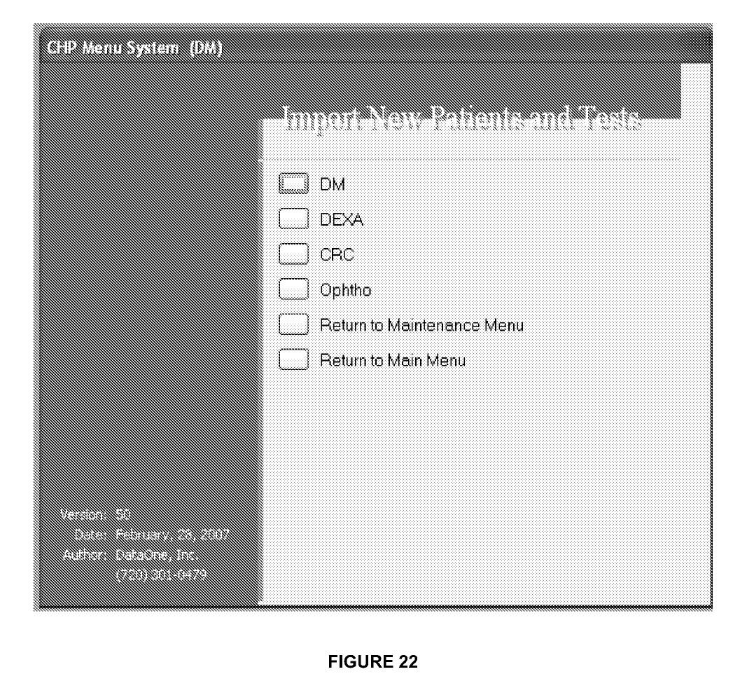

FIG. 22 is a screen shot of the Import New Patients and Tests screen, which is accessed by selecting the Database Maintenance screen shown in FIG. 21. This screen allows the user to import new patient information for a specific medical service, such as DM (diabetes), DXA (bone densitometry) CRC (colorectal cancer screening), and Opththo (eye examinations). The import data is saved in a location specified in the Manage System Information—Tests screen, which can be accessed from the database Menu Management screen shown in FIG. 21. This screen also allows the user to select Return to Maintenance Menu, which accesses the Database Menu Management screen shown in FIG. 21, and Return to Main Menu, which accesses the Main Menu shown in FIG. 3.

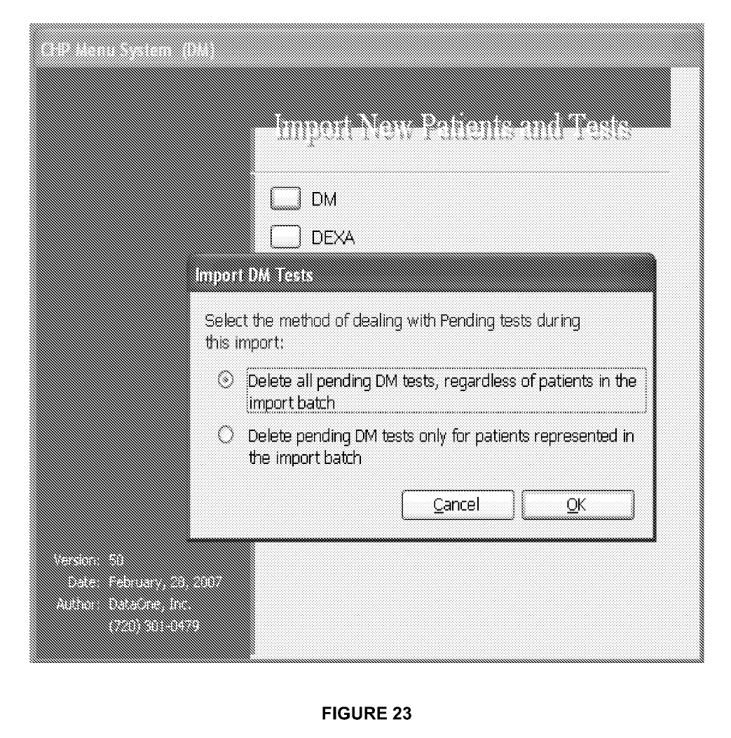

FIG. 23 is a screenshot of a selection box that appears when DM is selected on the Import New Patients and Tests screen of FIG. 22. This screen allows the user to import new patient records into the tracking system either by deleting, for the specified service, all records corresponding to patients who have not yet received outreach or by leaving intact all patient records already in the system while appending or overwriting any new information within the current import batch.

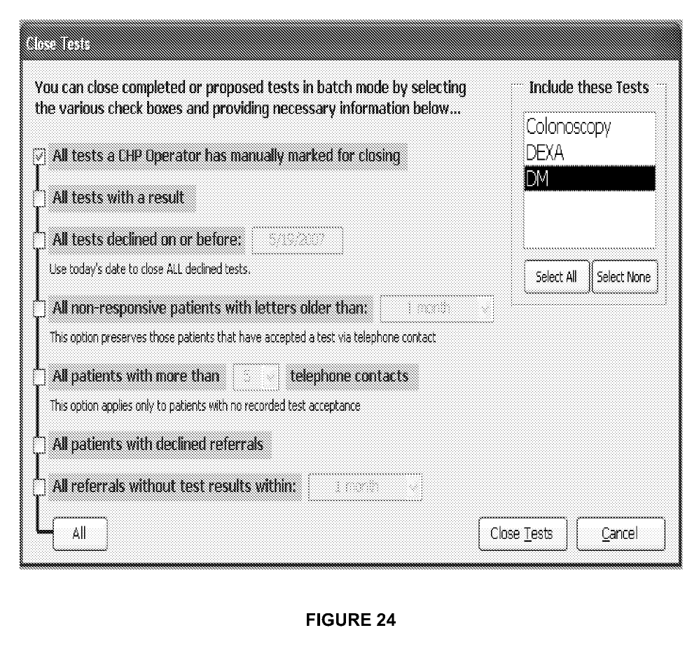

FIG. 24 is a screenshot of the Close Tests screen, which can be accessed from the Menu System screen shown in FIG. 21. This allows the user to close, i.e., to specify that the outreach process has gone as far as possible and that the tracking process has been completed, all patients and tests meeting specified criteria, such as those which have been manually marked for closing, those with a result, those declined on or before a certain date, those for patients who did not respond to letters older than a specified date, those for patients with more than a specified number of telephone contacts, patients who decline services, and tests that have not had results logged within a specified period of time.

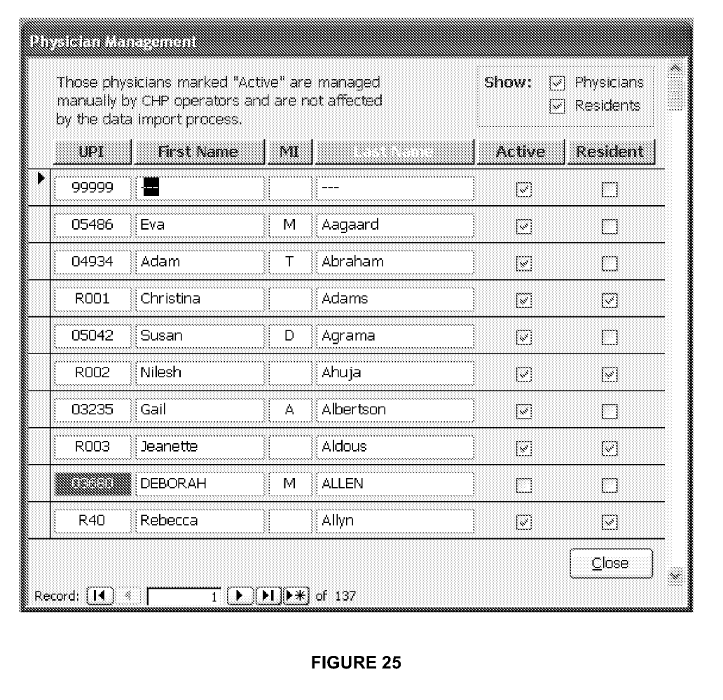

FIG. 25 is a screenshot of the Physician Management screen, which is accessed by selecting “Manage Physicians” on the Menu System screen shown in FIG. 23. This screen allows the user to specify and view physicians and residents who are active in the system.

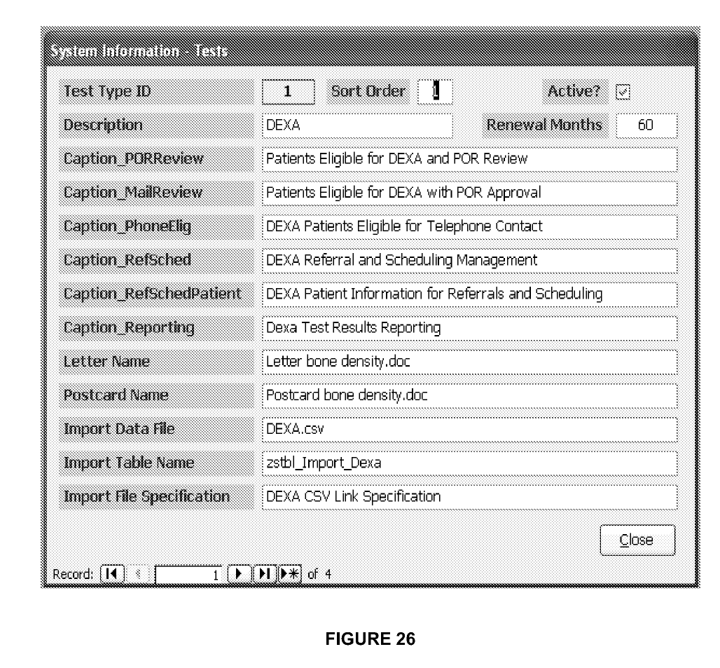

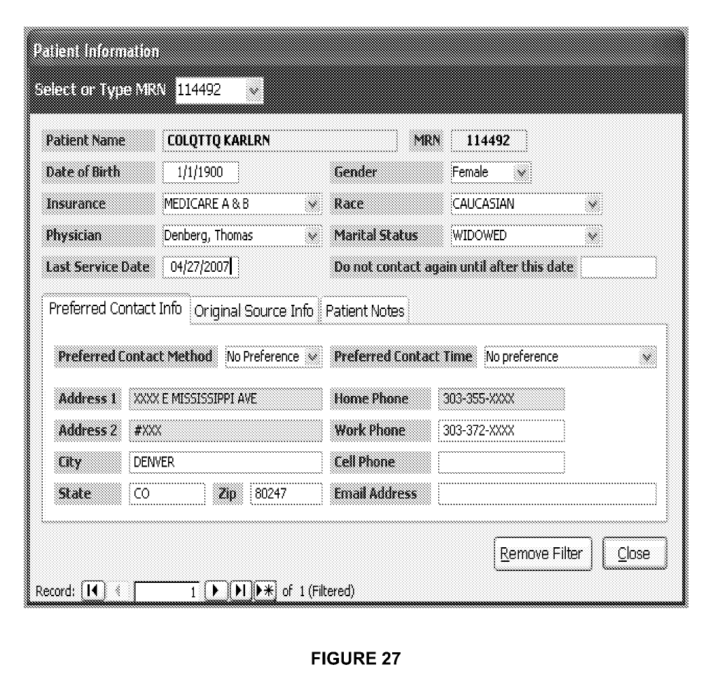

FIG. 26 is a screenshot of the System Information Management screen accessed by selecting “Manage System Information—Tests” on the Database Maintenance screen shown in FIG. 21. This screen allows the user to specify which medical services (tests) are being scheduled by the system and edit naming and locations of import files and letter/postcard templates. FIG. 27 is a screenshot of the Patient Information screen, which can be selected on the screen shown in FIG. 3. The patient information (search by MRN) screen lists patient information for all patients in the system sorted by Medical Record number (a unique identifier). This screen provides identification, contact and demographic information for the patients. If the user double clicks on a patient in the eligibility or mailing screens; this same patient information will appear.

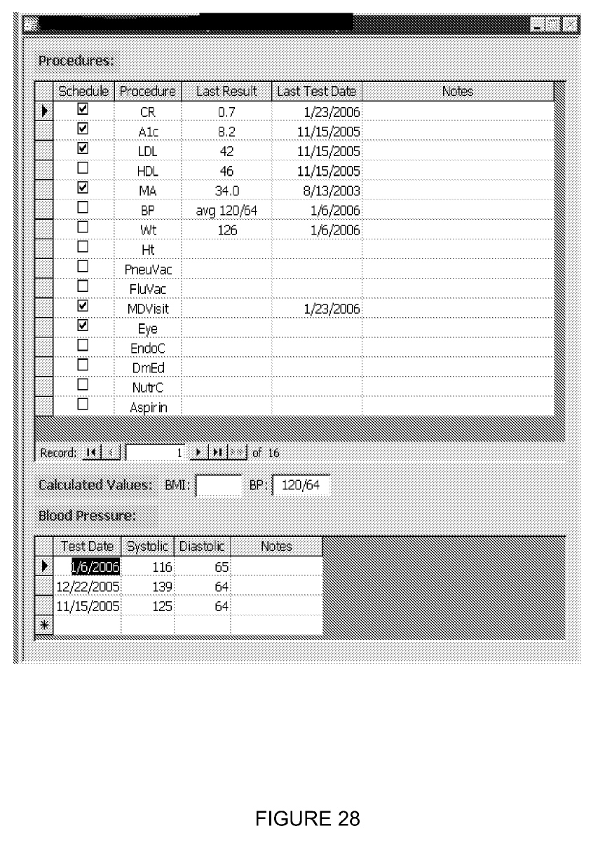

FIG. 28 is a screenshot summarizing diabetes services that an individual patient is eligible to receive. Initially, these services are flagged automatically by the computer based on published guideline criteria (e.g. American Diabetes Association). The operator has the ability to override these computer-set flags if this seems appropriate. This information can then be summarized in outreach letters to patients or for electronic review by medical providers, discussed with patients telephonically, and it can be used to help guide the HPOS operator in terms of scheduling appointment dates. This particular screen shot represents a way of organizing information to support an “in-house” (or within-health system) disease management program for diabetes. A similar concept can be applied to programs for asthma, chronic obstructive pulmonary disease, congestive heart failure, and other chronic conditions.

### EXAMPLES

We have established a HPOS at University of Colorado Hospital. Staffed by three outreach coordinators (3.0 FTE) and supporting six primary care practices with an aggregate of over 200,000 outpatients visits per year, the HPOS currently facilitates four primary prevention services: mammography for women over age 50, bone densitometry for women over age 65, prostate cancer screening for men over age 50, and colorectal cancer screening for men and women over age 50. HPOS also offers a set of chronic disease services for diabetes: PCP visits, retinal exam, and laboratory assessments at recommended intervals or when indicated because patients are not meeting guidelines for blood pressure, lipid control, and aspirin use, as well as education classes and endocrinologist assessments for patients with poor glycemic control.

### Example 1

### Bone Densitometry

Tracking of patients for bone densitometry (DXA) screening is used to illustrate the functionality of the HPOS. Initially, PCPs authorize HPOS to arrange, on their behalf, DXA exams for any of their patients who are eligible. Administrative data queries yield a set of patient records corresponding to women over age 65 who have not completed a DXA exam within UCH. These records are imported into the HPOS information management system. Invitation letters are then generated and mailed to eligible patients. These letters mention the name of the patient's PCP, summarize the United States Preventive Services Task Force (USPSTF) recommendations and rationale for DXA, and encourage women to contact the HPOS call center in order to arrange for an exam. Each letter is accompanied by a postage-paid return postcard on which recipients can indicate whether they previously had a DXA exam outside of UCH (and the results, if known), no longer receive their primary care within UCH, are uninterested in DXA, or would like the HPOS to call them at a given time and phone number. If a patient does not respond to the letter within two weeks, either by postcard or a telephone call, an HPOS coordinator makes outcalls to the patient's home, leaving a voice message on the first attempt. Once telephonic contact is established, the coordinator reviews the recommendations for DXA and, if the patient accepts, schedules an exam over the phone using the computerized radiology scheduling system. An antecedent provider visit is not required. A reminder postcard noting the date, time, and location of the exam is then mailed to the patient. At the same time, the coordinator uses the EMR to request a DXA authorization from the PCP. In this way, the electronic DXA order originates with the PCP (for billing and compliance), the DXA results return directly to the PCP (for appropriate clinical follow-up), and the transaction is well documented in the EMR. For any needed follow up, PCPs decide on a case-by-case basis whether to convey their recommendations in face-to-face visits, by mail, or over the phone. A summary report of the outreach process, including all communication attempts and whether a patient accepted an exam, is generated and included in the EMR. After the DXA exam is completed, the results are logged for reporting purposes and to facilitate clinical and financial analyses. As previously described, the above process is coordinated and tracked by our HPOS information management system.

To evaluate the performance of a patient recall intervention that relies on an outreach coordinator with a bachelor's degree to prompt women by mail and telephone about their eligibility for bone densitometry (DXA) screening and allow them to schedule an exam without a medical provider visit ahead of time 564 women age 65-79 years at average risk for osteoporosis without a history of DXA were tracked to determine rates of DXA completion and the change in proportion of screened women during a seven month intervention period; case finding for clinically significant bone loss; frequency of appropriate clinical follow-up; DXA no-show rates compared with usual care; and clinician satisfaction.

Through patient recall, rates of DXA screening rose significantly (p<0.0001) and the proportion of the eligible clinic population screened increased by 13%. Thirty percent of patients had clinically significant bone loss with almost all of these receiving follow-up. DXA no-show rates were comparable to usual care, and provider acceptance was high. The patient recall intervention substantially increased DXA screening, allowing pharmacologic therapy to be started much earlier in some women with significant bone loss. It imposed minimal burden on providers and enhanced patient convenience. This type of program has utility for additional preventive services.

Substantial disability, mortality, and direct care costs (more than 18 billion dollars in 2002) are associated with osteoporotic fractures. One in two women over the age of 50 will eventually have an osteoporosis-related fracture and 22% of 75-year-old women with osteoporosis will have a hip fracture within ten years. In order to minimize irreversible bone loss and reduce fracture risk, the United States Preventive Services Task Force (USPSTF) recommends that average risk women should complete dual-energy x-ray absorptiometry (DXA) screening starting at age 65. Nonetheless, the test is underutilized, especially among older women.

The delivery of preventive services such as DXA screening is suboptimal in ambulatory clinic populations (Yarnall, K S, et al. (2003)). Clinic visits are often too brief for medical providers to review and arrange for all recommended preventive care. Providers are frequently hampered by poorly-organized clinical data and a lack of automated reminders. Many patients do not know about preventive care recommendations and prefer to focus on acute concerns during clinic visits.

In breast cancer screening, the primary care office-based use of mailed patient reminders and personalized telephone outreach, in combination with the direct scheduling of mammography by patients rather than primary care providers (PCPs), has mitigated many of these barriers. Such mechanisms, allowing patients to bypass face-to-face visits with PCPs in order to obtain testing, improve mammography completion.(Valanis, B G et al. (2002); Taplin, S H, et al. (2000), King, E S, et al. (1994)). We developed a health promotion outreach system to improve the delivery of preventive and chronic disease care in the outpatient setting (Denberg, TD et al. (2008)). Using this system, an outreach coordinator with a bachelor's degree contacted patients in a large ambulatory general medicine practice. The coordinator alerted patients by mail and phone about the importance of DXA screening and allowed them to directly schedule DXA exams without seeing a PCP, but nonetheless kept PCPs informed and involved throughout the process. We hypothesized that this intervention would be associated with a significant increase in the rate of

This study was carried out in a large ambulatory general internal medicine practice affiliated with the University of Colorado Hospital. The practice provides primary care to a diverse patient population (approximately 50,000 visits per year) and is staffed by 27 attending physicians, four nurse practitioners, and 12 primary care residents.

The U.S. Preventive Services Task Force (USPSTF) recommends that women without other risk factors begin screening for osteoporosis starting at age 65. Women were eligible for outreach if they did not have a prior administrative claim for a DXA exam (CPT codes 76075-76077, 77080-77082) within the health system, had seen a PCP in the practice at least once in the preceding 18 months, and were between the ages of 65 and 79 years. The overwhelming majority of women in this age range are enrolled in Medicare and eligible for Medicare reimbursement for this procedure. Women over the age of 79 were excluded based on PCP preferences that these women receive DXA referrals through face-to-face clinic visits because they are more likely than younger women to have complicated comorbidities. Based on a review of the Electronic Medical Record (EMR, Allscripts v.10, Chicago, Ill.), women were excluded for outreach, regardless of DXA history, if they had clinic notes suggesting an active cancer or terminal diagnosis, were currently taking a bisphosphonate, were deceased, or no longer appeared to be receiving care within the system. The cohort of DXA-eligible patients varied over time. Women became eligible for DXA as they turned 65 years old or entered the system for the first time by establishing a relationship with a PCP. Other patients became ineligible after they turned 80 years old or if they had not seen their PCP in clinic in over 18 months.

All PCPs pre-authorized the intervention team to arrange DXA exams for eligible patients. Administrative data queries (updated on a monthly basis) identified women between the ages of 65 and 79 who appeared to be eligible for DXA. These records were imported into an information management utility that was developed for this and other prevention and chronic disease outreach interventions. An outreach coordinator (OC) with a bachelor's degree then spent an average of three minutes per patient reviewing the EMR to exclude women from this list who did not meet eligibility requirements that could not always be evaluated automatically (e.g. deceased, moved, DXA result from an outside facility scanned into the record). The information management utility generated an invitation letter that was mailed to each patient, which included the name of the patient's PCP, summarized the USPSTF recommendations and rationale for DXA, and encouraged the patient to contact the call center to arrange an exam. Each letter was accompanied by a postage-paid return postcard on which the patient can indicate whether she previously had a DXA exam outside of the health system (and the results, if known), no longer received primary care within the system, was uninterested in DXA, or preferred for us to contact her at a specified time and phone number. If the patient did not respond to the letter within two weeks by postcard or telephone, the OC made up to three calls to her home at different times of the day over a period of eight weeks, leaving a voice message on the first attempt. If a patient could not be reached within an eight week period, she was regarded as a passive decliner of a DXA exam. If telephone contact was established, the OC reviewed the recommendations for DXA and, if the patient accepted, the OC scheduled an exam over the phone. The OC then requested DXA authorization from the PCP by means of the EMR system. This process assured that the electronic DXA order originated with the PCP (for billing and compliance), that DXA results were returned directly to the PCP (for clinical follow-up), and that the transaction was documented in the EMR. After the order was approved, the OC mailed a reminder postcard to the patient noting the date, time, and location of the exam. After receiving test results, PCPs decided on a case-by-case basis whether to convey their follow-up recommendations to patients through face-to-face visits, by mail, or over the phone. The call center was open between the hours of 8 AM and 7 PM, Monday through Thursday; at other times patients could leave messages requesting a callback.

In usual care, patients receive referrals for DXA during face-to-face clinic visits with PCPs. In this study, the outreach intervention and usual care operated concurrently within the same practice setting.

In order to identify and resolve operational difficulties, we carried out a one month phase of intervention implementation and pilot-testing that involved outreach to six patients. Because this number was very small and the intervention was extremely limited in scope, we counted the months six months preceding beginning of the test as part of the pre-intervention period. We then carried out a comprehensive outreach process over five months, after which we discontinued outreach. An intervention follow-up period spanned the two months following the test, during which DXA exams scheduled in the preceding months (through both usual care and outreach) were completed and tracked. The comprehensive outreach and follow-up intervals together comprised the intervention period.

Two primary outcomes were assessed and several process measures as follows:

1. The baseline and post-intervention follow-up change in the proportion of eligible women who completed DXA screening. We used administrative data to determine, as a snapshot, the baseline proportion of eligible women who were up-to-date with screening. A similar procedure allowed us to determine the proportion of women up-to-date with screening at the end of the two-month intervention follow-up period. The change in the proportion of the population up-to-date with screening was calculated as the difference between these two proportions. We then calculated more accurate baseline- and intervention follow-up proportions by excluding women from the administrative-claims-based denominators if they were found to be ineligible based on manual EMR review or outreach (e.g. women returned a postcard or informed us by telephone that they previously completed DXA outside the health system). Finally, an intervention-specific effect on the change in the proportion of women up-to-date with screening was estimated by dividing the number DXA exams completed through outreach by the mean number of eligible patients in the baseline and post-intervention follow-up denominators.

2. Changes in the DXA completion rate before, during, and after the outreach period. Using a time series evaluation (described in Statistical Methods, below), we calculated monthly rates of DXA completion over a 12-month pre-intervention period and an approximately seven month intervention period that comprised active outreach and follow-up. An intervention effect was assumed only if a woman completed an exam scheduled through the outreach process; usual care was assumed for all other completed exams.

### Process Measures

1. Number and proportion of women screened through the intervention whose DXA results demonstrated normal bone density, mild osteopenia (T score −1.1 to −1.9), advanced osteopenia (T score −2.0 to −2.4), and osteoporosis (T score≦−2.5).

2. Based on manual EMR review of clinician notes (about three minutes per patient), the proportion of women who completed DXA through outreach who subsequently initiated therapy with a bisphosphonate and/or Vitamin D/calcium, completed vitamin D testing, received a referral to endocrinology, or received communication about their test results or bone loss prevention from a PCP.

3. The number, proportion, and sociodemographic characteristics of women who scheduled and refused a DXA exam as a result of outreach.

4. Rates of intervention DXA appointment non-adherence compared with usual care. The IDX radiology scheduling system was used to identify women in the practice who had been scheduled for a DXA exam over the intervention period and then coded as non-adherent those who, within four weeks of their scheduled date, did not have DXA results available in the EMR.

5. Clinician satisfaction with the intervention. A brief, anonymous, online survey was devised which was administered to attending PCPs in order to assess their opinions about the mechanics of the intervention, its effects on relationships with patients, and their ability to provide clinical follow-up for women who completed screening. Respondents indicated their level of agreement with several statements using a five-point Likert response format (“agree strongly” to “disagree strongly”). These items were followed by an open-ended solicitation of general feedback.

Statistical procedures were carried out using SAS (version 9.1, SAS Institute, Cary, N.C.). The unadjusted proportions of the eligible population who had completed a DXA exam immediately before and after the intervention were considered. A time series evaluation was also carried out with monthly time points that assessed the interruption (immediate effect) and change in slope related to activating the intervention. To do this, a regression model was derived based on the total number of completed monthly DXA exams on time (month) with a shift in intercept and slope (allowing for curvature) during the intervention. It was expected that a strong intervention effect would overcome the lack of data points to build a reliable regression model and used an autoregressive of order 1 (AR(1)) to account for autocorrelation among the data for DXA exams. Chi-square tests were used to determine the strength of association between sociodemographic characteristics (age, race/ethnicity, and marital status) and willingness to schedule an exam, and to compare the proportion of women who were non-adherent through usual care with those who were non-adherent through the intervention.

Proportion screened: Based only on administrative data, 57.9% (1,085/1,873) of the eligible clinic population had completed DXA screening at baseline. During the intervention period, the numbers of women who completed DXA by means of outreach and usual care were 244 and 230, respectively. As a combined result of these exams, 75.2% (1,559/2,072) of the population had completed screening by the end of the intervention period. After removing from the baseline and follow-up denominators 91 patients who were deemed ineligible based on manual EMR review and information obtained through outreach (e.g. previously completed DXA outside of the system, deceased, moved), the revised proportions screened at baseline and post-intervention were 60.9% (1,085/1,782) and 78.7% (1,559/1,981), respectively. Ultimately, the percentage increase in the population screened attributable to the intervention (as opposed to usual care) was approximately 13.0 percentage points (244 divided by the average of 1,782 and 1,981). Table 3 displays the characteristics of the 564 patients who received outreach and were eligible for DXA. The proportions eligible after outreach that scheduled and then actually went on to complete DXA exams were 49.8% and 43.3%, respectively.

TABLE 3
Characteristics and DXA Scheduling Outcomes of Patients
Eigible for DXA after Outreach*

| | Total Accept | | Total Refuse | | |
|---|---|---|---|---|---|
| Characteristic | N | % | N | % | p^{†} |
| Total = 564 | 281 | 50% | 283 | 50% | |
| Age | | | | | 0.12 |
| 65-69 n = 254 (45%) | 136 | 54% | 118 | 46% | |
| 70-74 n = 187 (33%) | 93 | 50% | 94 | 50% | |
| 75-79 n = 123 (22%) | 52 | 42% | 71 | 58% | |
| Race | | | | | 0.01 |
| Non-Latino white n = 279 (49%) | 158 | 57% | 121 | 43% | |
| African American n = 73 (13%) | 26 | 36% | 47 | 64% | |
| Latino n = 26 (5%) | 15 | 58% | 11 | 42% | |
| Asian n = 28 (5%) | 14 | 50% | 14 | 50% | |
| Other/Unknown n = 158 (28%) | 68 | 43% | 90 | 57% | |
| Marital status | | | | | 0.29 |
| Married n = 269 (48%) | 141 | 52% | 128 | 48% | |
| Not married n = 138 (24%) | 61 | 44% | 77 | 56% | |
| Widowed n = 115 (20%) | 61 | 53% | 54 | 47% | |
| Unknown n = 42 (7%) | 18 | 43% | 24 | 57% | |

*An additional 91 patients were ineligible for DXA based on information obtained during outreach.
^{†}p-values correspond to chi-square tests for categorical variables to detect differences within strata of each patient characteristic.

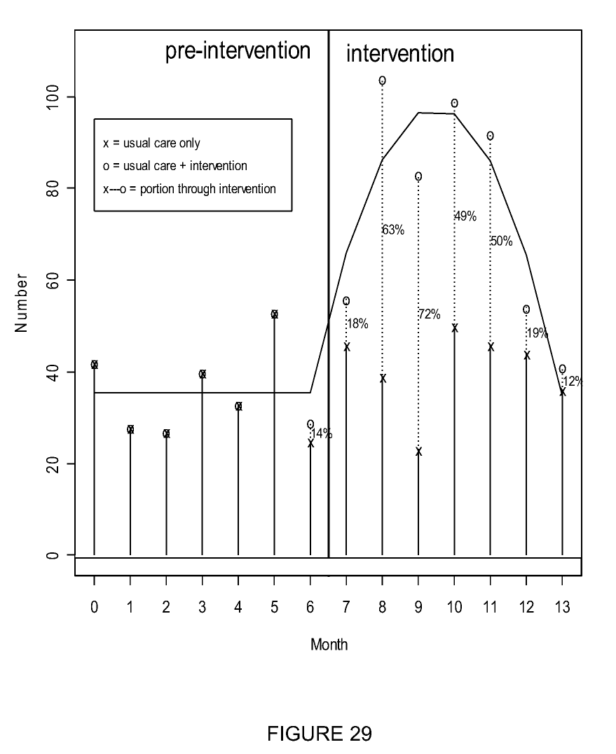

Rate of DXA completion: FIG. 29 is a graphical depiction of DXA volume during the intervention period compared with usual care. Quadratic function=35.5+35.6*(Month−6)*(Month−6) and estimated AR(1) parameter=−0.4. A regression line for total DXA volume is overlaid. As anticipated, there was a non-linear significant slope change (p<0.0001) after the intervention was activated, with the rate of DXA completion rising dramatically, stabilizing during the third and fourth month of the test and then falling near baseline after the outreach process was halted. The average month-to-month number of DXA exams before the intervention was not significantly different from the average usual care number during the intervention period (41 vs. 36, p=0.33), suggesting that the ongoing intervention did not lead providers to increase their rates of in-clinic DXA referrals (i.e. contamination was not evident).

Case-finding: Table 4 summarizes the T-score results of the 244 completed DXA exams. Initiation of pharmacologic therapy was indicated for 30% of these patients based on National Osteoporosis Foundation (NOF) criteria (T-score≦−2.0) for average-risk women¹¹, and for 13% on the basis of World Health Organization (WHO) criteria (T-score≦−2.5).

TABLE 4
Bone Densitometry Results (T-scores) and Clinical
Follow-up of Patients Who Completed Screening

| Condition (t-score) | N | % | Clinical follow-up |
|---|---|---|---|
| Osteoporosis (≦−2.5) | 31 | 13% | 94%* |
| Osteopenia (−2.0 to −2.4) | 41 | 17% | 78%^{†} |
| Osteopenia (−1.1 to −1.9) | 88 | 36% | 100%^{‡} |
| Normal (≧−1.0) | 84 | 34% | 88%^{§} |
| Total | 244 | 100% | |

*All patients received follow-up contact; one patient never scheduled and one patient cancelled a recommended follow-up PCP appointment.
^{†}Four patients had no follow-up contact (three of these were patients of medical residents); five never scheduled a recommended follow-up PCP appointment.
^{‡}20% random sample.
^{§}In 20% random sample, 2/17 patients had no follow-up contact.

Clinical follow-up: As summarized in Table 4, the PCP or a clinic representative contacted all 31 patients with osteoporosis in order to recommend a follow-up appointment with the PCP and/or to facilitate education, vitamin D testing, endocrinology referral, or the initiation of medical therapy. Two months after the intervention was completed, all patients with osteoporosis received follow-up except for two patients who did not complete a recommended follow-up PCP appointment. Among patients with advanced osteopenia, all but four patients (three of whom had resident physicians) received no follow-up contact while five more did not schedule a recommended follow-up PCP appointment. A 20% random audit of patients with mild osteopenia and normal bone density also revealed high rates of follow-up contact by the clinic.

TABLE 5
Reasons for DXA Refusal

| Reason | N | % |
|---|---|---|
| Not interested/other* | 124 | 33% |
| Will wait to discuss during a PCP visit | 61 | 16% |
| No transportation | 7 | 2% |
| No contact could be established | 91 | 24% |
| Already had test | 53 | 14% |
| No longer a health system patient (includes | 38 | 10% |
| deceased) | | |
| Total | 374 | |

*Most commonly-cited reasons in this category included personal health issues, caring for an ill family member, and traveling out of town.

DXA appointment non-adherence: The proportion of women who did not attend scheduled exams was 13% in the intervention group (244 completed out of 281 accepted) and 16% in the usual care group (284 completed out of 338 scheduled), a non-significant difference (p=0.18).

PCP feedback: Twenty-eight of 31 PCPs shared their opinions about the intervention through an anonymous online survey. Table 6 summarizes responses to survey items. Overall, PCP attitudes were very favorable. Illustrative comments included: “Patients have been pleased with the intervention,” “I think the intervention helps build a relationship with my patients because they know we care about their health even between MD visits,” “It is terrific; we need more of these automatic items,” “Overall, an excellent option. It helps ‘fill in the gaps’ when there are things that just can't be covered in a routine visit.” A small number of PCPs did voice concerns. For example, one PCP remarked, “I have had at least 2 patients tell me they would not want anyone other than me ordering their tests, and they refused on the phone and then wanted to get my opinion before going through with one.” The most common concern had to do with the perceived ability to provide appropriate follow-up. One PCP wrote, “I have very limited appointment availability right now. The work per patient to do counseling and follow-up over the phone for DXA results is significant.” Another commented, “If I get an abnormal DXA result, I mail them a copy and ask them to schedule an appointment to discuss treatment options. I let them know it is not urgent, but I find a face-to-face visit is much better if we have not already discussed it.”

DXA accepters/decliners: African American women were less likely than women of other racial/ethnic groups to accept an exam (Table 3). Of all women who accepted, 36% did so by calling the scheduling coordinator, while the remaining 64% accepted after the coordinator contacted them. Table 3 summarizes the primary reasons for DXA refusal, illustrating that among patients who were otherwise eligible and could be reached, lack of interest, competing priorities, and a desire to first discuss the exam with a PCP were most common.

TABLE 6
Primary Care Provider Opinions Regarding Intervention (n = 28)*

| | Agree^{†} | Neutral | Disagree |
|---|---|---|---|
| Survey Item | N (%) | N (%) | N (%) |
| 1. DXA (bone densitometry) scans being offered to | 1 (4%) | 5 (18%) | 22 (78%) |
| patients over the phone by a nurse or clinical assistant | | | |
| without my direct involvement has compromised the | | | |
| quality of my relationship with these patients. | | | |
| 2. As long as I authorize DXA scan orders entered by a | 25 (89%) | 1 (4%) | 2 (7%) |
| nurse or clinical assistant and receive the results, | | | |
| eligible patients do not need to have a face-to-face | | | |
| visit with me in order to discuss and receive referrals | | | |
| for osteoporosis screening. | | | |
| 3. I believe that the intervention has made a meaningful | 21 (75%) | 7 (25%) | 0 (0%) |
| difference in terms of ensuring that a larger proportion | | | |
| of older female patients in my panel receive | | | |
| osteoporosis screening. | | | |
| 4. I have had difficulty arranging follow-up (e.g. | 5 (18%) | 6 (21%) | 17 (61%) |
| calcium/vitamin D counseling, initiating | | | |
| bisphosphonates) based on the results of DXA scans | | | |
| scheduled by the intervention team. | | | |

*Items in bold indicate an unfavorable opinion about the intervention.
^{†}Agree and Disagree combine “(dis)agree strongly” and “(dis)agree somewhat” in the original survey.

An outreach intervention that allowed patients to directly schedule DXA exams over the phone without seeing a PCP ahead of time for a referral, but that nonetheless kept PCPs informed and involved, quickly and substantially reduced the proportion of older women patients who had not yet completed bone densitometry testing in a general medicine practice. About 50% of women scheduled and 43% completed DXA screening as a result of outreach. A large proportion (30%) of screened women had clinically significant bone loss, defined as a T-score≦−2.0, and a majority of these received clinical follow-up. Non-adherence with scheduled DXA procedures was comparable to usual care. A large majority of PCPs had favorable opinions about the intervention and many would like to see it used for other types of preventive services.

It is important to detect and initiate treatment for osteoporosis as early as possible. Two large trials have shown that a bisphosphonate produces a very substantial reduction in the risk of vertebral and non-vertebral fractures within one to three years (Black, D M et al. (1996, Harris, S T et al. (1999)). Another large trial identified a 40% reduction in hip fracture risk for osteoporotic women age 70-79 after three years of bisphosphonate therapy, and substantial benefit was already evident within six to 18 months of starting therapy (McClung, M R, et al. (2001)). Given the high prevalence of clinically significant bone loss in the population contacted through the outreach program, an intervention that identifies this condition even one year earlier than would have been achieved through usual care is beneficial. By markedly increasing the rate at which the proportion of the eligible population completed screening, many women in our clinic were diagnosed with clinically-significant bone loss years earlier than would have been achieved through usual care.

Mailed outreach followed by telephone contact and the direct scheduling of mammography substantially boosts rates of breast cancer screening. A similar strategy improved DXA screening in our ambulatory clinic population. We identified a prior study in which physician prompts and patient mailings—but no telephone contact—was associated over a five month period with an 18.1% higher yearly rate (28.9% vs. 10.8%) of DXA completion compared with usual care (Lafata, J E et al. 2007)). A second study used an Interactive Voice Response (IVR) system to encourage DXA among health plan members, but only 0.2% of patients scheduled an exam (Polinski, J M et al. (2006)). Another study published in abstract form only reported that 10%-20% of high-risk patients (smokers with a low body mass index) completed DXA after they were targeted by mail, no phone calls were made, and PCP visits were required for referrals (Binstock, M. (2001)). Finally, a second abstract reported that when women aged 65 and over received a letter followed by a phone call, and the direct scheduling of DXA was permitted, 39.6% completed screening through this process compared with 13.2% who received usual care. (Ayoub, W T et al. (2007)). This was comparable to the 43.3% observed in the present study that utilized a similar approach.

In a preliminary study, nearly 50% of the patients in the general medicine practice indicated that they rarely or never see a PCP in order to discuss prevention (Denberg, T D, et al. (2007)). Furthermore, over 70% expressed interest in the type of preventive healthcare delivery model that was implemented for DXA screening. These favorable attitudes appear to have been confirmed in practice. Although uninformed about the intervention ahead of time, 43% of contacted patients completed a DXA exam. At the same time, the preliminary study of patient attitudes also suggested that socioeconomically disadvantaged patients can be more reticent than others about this type of outreach, and in the present study it was noteworthy that African American women accepted a DXA exam less frequently than all other racial/ethnic groups. Future applications of this type of intervention might benefit from more in-depth assessments and targeted approaches to racial/ethnic and socioeconomic barriers to outreach.

Although PCPs were concerned about their ability to provide follow-up for intervention-screened patients who had clinically significant bone loss, they nonetheless did provide counseling, further testing, or initiation of medical therapy in the vast majority of such cases. When follow-up did not take place, this was most often attributable to patient non-adherence with a written or telephonic recommendation from the clinic to schedule a follow-up appointment or to the lack of follow-up communication by resident (rather than attending) physicians. The communication of test results and treatment recommendations by a clinician is a strong predictor of initiating bisphosphonate and calcium therapy, (McLeod, K M et al. (2007), Fitt, N S, et al. (2001), Brennan, R M et al. (2004)). Outreach coordinators help to improve clinical outcomes by sending post-DXA educational materials to patients and helping them to schedule follow-up appointments with their PCPs.

In addition to other benefits, this type of program increases clinical efficiency by making bone density testing more convenient for patients. It also frees up time in overloaded office visits to discuss other health concerns. Finally, it supports new federal quality reporting initiatives based on the percentage of female patients aged 65 years and older who have a central DXA measurement ordered or performed at least once since age 60 or pharmacologic therapy prescribed within 12 months.

This study had several limitations. The intervention was carried out in a single academic healthcare setting that is not representative of other types of practice environments. The bulk of the intervention was carried out over the coldest months of the year and the average number of usual care DXA exams remained unchanged prior to and during the intervention; thus, a seasonal effect is unlikely to have influenced the final results. Because the study was designed in collaboration with a hospital to demonstrate the feasibility and benefits of this approach to preventive care, operational needs precluded a randomized, controlled trial. Nonetheless, the study had several significant strengths as a quality improvement initiative including multiple pre- and post-intervention measurements, a combination of quantitative and qualitative assessment, and the measurement of relevant outcome and process measures.

In conclusion, we were able to demonstrate that a patient recall program directed by an outreach coordinator with a bachelor's degree not only increased rates of bone densitometry testing in an ambulatory population, but that patient and provider acceptance was high and clinical follow-up was excellent. This model can also be used for other types of preventive health services.

### Example 2

### A Patient Outreach Program Between Visits Improves Diabetes Care

The objective of the study was to reduce barriers to guideline-based diabetes care, which include poor patient activation, haphazard clinic appointments, poorly organized medical records, and a lack of automated physician decision support. We developed a patient recall intervention to mitigate these barriers and improve diabetes care coordination. We evaluated this intervention in terms of operational feasibility, provider and patient acceptance, and effects on process-of-care measures.

Based on American Diabetes Association criteria, we identified patients with diabetes in a large internal medicine practice who were due for provider visits; hemoglobin A1c, lipid, microalbumin, and serum creatinine laboratories; and retinal examinations. An outreach coordinator contacted patients to summarize this information and schedule recommended services. We assessed patient responsiveness to outreach and used medical chart review to compare diabetes care rendered before and after the intervention. Providers provided feedback about their satisfaction with the program.

Results: Over 3 months, 709 patients were overdue for diabetes-related services. Of 415 overdue for provider visits, a total of 125 (30.1%) completed such visits arranged by an outreach coordinator and, of these, 101 (80.8%) completed laboratories at least a day ahead of time. An additional 52 out of 415 patients (12.5%) bypassed the outreach coordinator to self-schedule diabetes visits within a six week period after the outreach letter was mailed. Among overdue patients, completion of recommended services and intensity of diabetes care were significantly greater through the outreach program compared with traditional care. Provider attitudes were favorable.

The outreach intervention was associated with improved timeliness and intensity of diabetes care in an outpatient setting.

Guidelines for care promulgated by the American Diabetes Association (ADA) (American Diabetes Association. Standards (2007)) specify intervals for receiving diabetes-related medical services in order to promote glycemic control and minimize complications. Nonetheless, large numbers of patients with diabetes in ambulatory clinic populations are not up-to-date with guideline-based evaluations. In outpatient settings, the delivery of chronic disease services is suboptimal (Kabcenell A, et al. (2006); Ostbye T, et al. (2005); Yarnall K S, et al. (2003)). Clinic visits are often too brief for medical providers to review and arrange for all recommended care. Providers are frequently hampered by poorly-organized clinical data and a lack of automated reminders. Most patients do not know about recommendations for care and tend to focus on acute concerns during clinic visits. Finally, many patients fail to make clinic appointments or do so for reasons unrelated to their chronic illness.

To overcome many of these barriers we developed a health promotion outreach system (Denberg T D, et al. (2008)). The purpose of this system is to increase access to and enhance the delivery of guideline-based care by communicating with patients outside of clinical settings. Our diabetes-specific outreach system incorporates: (a) electronic queries of administrative claims to identify patients who are not up-to-date with recommended diabetes services; (b) an outreach coordinator with a bachelor's degree who provides mail and telephone outreach to alert patients about recommendations for care and facilitate the immediate scheduling of services; and (c) to improve visit planning, advance provider notification about the intended diabetes focus of appointments arranged through the outreach process.

The purpose of this pilot study is to assess whether a novel outreach intervention is operationally feasible, improves the timely receipt of diabetes-related medical services as well as clinical attention to diabetes during primary care visits, and is acceptable to providers.

### Methods

Study setting. This study was carried out in a general internal medicine practice affiliated with the University of Colorado Hospital. The practice provides primary care to a diverse patient population (approximately 50,000 visits per year) and is staffed by 27 attending physicians, four nurse practitioners, and 12 primary care residents.

### Diabetes-Related Medical Services

Based on ADA criteria (1), we developed an intervention focused on achieving patient adherence with at least two hemoglobin Al c determinations and single urine microalbumin and serum creatinine laboratory yearly. The intervention also promoted the ADA's primary recommendation of retinal exams and lipid profiles on a yearly basis. Finally, although not an explicit ADA recommendation, clinic appointments were recommended to patients who had not seen their provider in over six months, because as a practical matter it is difficult to allocate clinician time to reinforce disease management principles and review disease status and laboratories, including twice-yearly A1c laboratories, outside the context of such visits.

### Patient Population

To identify ongoing recipients of diabetes care within the clinic, patients were included in a patient registry if they had a record of at least two provider visits at any point in time associated with a 250.xx ICD-9 code within our billing claims system. In order to minimize outreach to patients no longer receiving care from one of our providers, we omitted patients whose last primary care visit was more than 18 months in the past. Because services could be scheduled 30 days into the future, patients were deemed eligible for outreach if they were coming due for at least one service during the next month. This means patients were excluded if they had an administrative claim for a primary care visit or hemoglobin A1c laboratory in the past five months (150 days) and a retinal exam, fasting lipid profile, and microalbumin/creatinine ratio within the past 11 months (330 days). Patients were also ineligible for outreach if they had a pending provider appointment within the next six weeks or had received an outreach letter during the previous 60 days. Supplementing this automated, claims-based algorithm, an outreach coordinator carried out a brief review of the electronic medical record (Allscripts Touchworks v. 10, Chicago, Ill.) to exclude patients who were deceased, had cancer or a terminal diagnosis, or were receiving ongoing diabetes care through an endocrinologist or within a nursing home.

### Intervention Description and Implementation

All providers pre-authorized the intervention team to identify and telephonically schedule recommended services for eligible patients. Administrative claims queries were updated on a monthly basis and patient records were imported into information management software that we developed for this and other prevention and chronic disease outreach interventions ((Denberg T D, et al. (2008)). The software generated patient invitation letters that included the name of each patient's provider, summarized the ADA recommendations, and encouraged patients to contact our call center to arrange for overdue services. Letters were accompanied by postage-paid return postcards on which patients could indicate whether they recently received services outside of our health system, no longer received primary care within our system, were uninterested in the recommended services, or preferred for us to contact them at a specified time and phone number. If patients did not respond to the letters within two weeks by postcard or telephone, the outreach coordinator made up to three calls to their homes, leaving a voice message on the first attempt. Patients who could not be reached within eight weeks were regarded as decliners of diabetes services. If telephone contact was established, the outreach coordinator reviewed the recommendations for care and, if patients accepted, the outreach coordinator immediately scheduled services including, whenever possible, laboratories at least a day (but preferably a week) before provider visits. The outreach coordinator then requested provider authorization for the identified services by means of the electronic medical record. This process assured that the electronic orders originated with the provider (for billing and compliance), that results were returned directly to the provider (for clinical follow-up), and that the transaction was documented in the electronic medical record. After the orders were approved, the outreach coordinator mailed reminder postcards to patients noting the date, time, and location of the services. The outreach coordinator then sent informational notes to providers notifying them via the electronic medical record of all scheduled services and the diabetes-specific purpose of pending provider appointments. The call center was open between the hours of 8 AM and 7 PM, Monday through Thursday; at other times patients could leave messages requesting a callback.

Following one month of pilot-testing, we conducted intervention outreach over three months after which we halted the process in order to evaluate outcomes that would inform a decision about whether to continue the program in the study clinic as well as deploy it in other clinics.

### Process Measures:

We assessed the following:

1. The number and proportion of patients in the diabetes registry who over the intervention period received outreach for provider visits and/or laboratory testing.

2. The number, proportion and sociodemographic characteristics of patients who completed provider visits arranged through outreach. A primary intervention effect was based on whether patients completed a provider visit scheduled by an outreach coordinator. Although outreach letters explicitly instructed patients to contact our call center, some patients bypassed this mechanism and instead called the clinic directly to schedule provider visits. Thus, we determined a secondary intervention effect based on whether patients self-scheduled and completed diabetes-related services within six weeks of the date invitation letters were mailed.

3. Among patients who completed provider visits arranged through outreach, the degree to which care associated with the intervention differed from traditional care these same patients had received most recently in the past three years when they were also overdue for a provider visit. Based on medical record review, we evaluated several aspects of diabetes care at two points in time. The first was care rendered during provider visits arranged through outreach and including follow-up visits that took place within one month afterwards (intervention-related care). The second was care rendered before implementation of the outreach intervention at a point when provider visits were most recently overdue and within one month afterwards (traditional care). Based on electronic medical record review of a random sample of 50 patients who completed outreach-scheduled provider visits, we compared intervention and traditional care in terms of the average number of days between overdue provider visits, average number of services for which patients were due, specific services obtained, and clinical actions related to diabetes documented in the electronic medical record, including referrals for diabetes education or endocrinology consultations; modification of antihyperglycemics, antihypertensives, and lipid-lowering agents; and provider review of diabetes-specific laboratory and physical exam findings. This type of traditional care comparison was warranted because only certain patients would be responsive to outreach and, using the best data available, the objective was to gain insight into whether care was improved for this group of patients.

4. Clinician satisfaction with the intervention. We devised a brief, anonymous online survey which we administered to providers in order to assess their open-ended opinions about the intervention's ease of use and mechanics, its effects on quality of care, and feedback received from patients.

### Statistical Methods

Statistical procedures were carried out using STATA (version 8.2, College Station, Tex.). We used chi-square tests to determine the unadjusted strength of association and multivariable logistic regression to determine the adjusted strength of association between sociodemographic characteristics (age, sex, race/ethnicity, type of insurance, and marital status) and completion of services, and to compare aspects of traditional care with the intervention among patients who responded to outreach. Variables whose level of significance in bivariate analysis was 0.25 or less were included in multivariable modeling (Hosmer D W, et al. (2000). Student's t-tests were used to compare the intervention and traditional care in terms of average numbers of laboratories due and average number of days overdue for provider visits.

### Institutional Review Board

This intervention was designed and carried out as a quality improvement program that relied on standard methods for creating patient registries and providing patient outreach. The Colorado Multiple Institutional Review Board approved publication of results following the removal of personal health information.

### Results

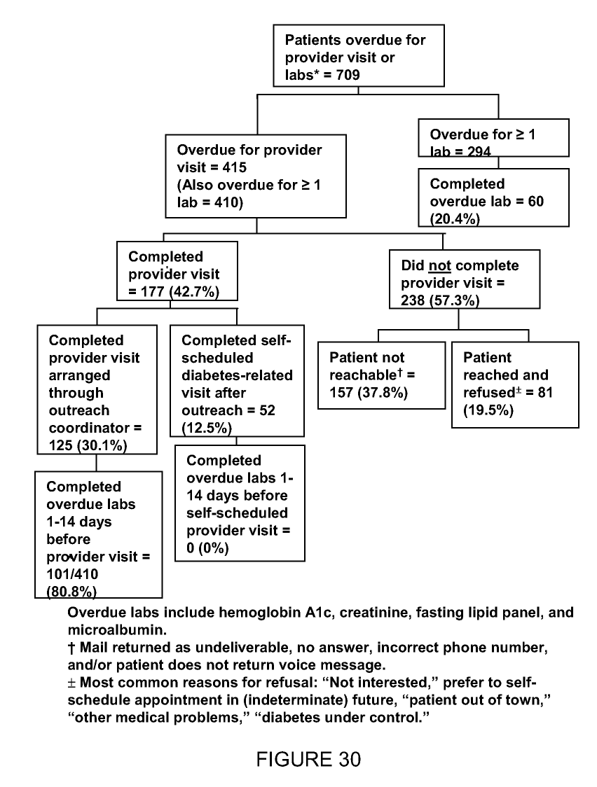

Outreach-eligible population and provider visit completion: FIG. 30 depicts the responsiveness of patients to outreach. Over a three month period, we identified 709 patients due for diabetes-related services (a monthly average of 18% of all patients in the diabetes registry). Of these, 415 were due for a provider visit (Table 7).

TABLE 7
Patients completing a provider visit through diabetes outreach

| | | N (%) needing and |
|---|---|---|
| | N (%) needing | completing provider |
| | provider visit | visit |
| TOTAL | 415 | 125 (30.1%) |
| Sex | | p = 0.02 |
| Female | 190 (45.8%) | 46 (24.2%) |
| Male | 225 (54.2%) | 79 (35.1%) |
| Age | | p = 0.06 |
| <50 | 60 (14.5%) | 9 (15.0%) |
| 50-59 | 118 (28.4%) | 38 (32.2%) |
| 60-64 | 78 (18.8%) | 23 (29.5%) |
| 65-74 | 105 (25.3%) | 34 (32.4%) |
| >=75 | 54 (13.01%) | 21 (38.9) |
| Marital status | | p = 0.08 |
| Married | 218 (52.5%) | 76 (34.9%) |
| Not married | 167 (40.2%) | 42 (25.2%) |
| Unknown | 30 (7.2%) | 7 (23.3%) |
| Race/ethnicity | | p = 0.49 |
| Black | 76 (18.3%) | 23 (30.3%) |
| White | 177 (42.7%) | 56 (31.6%) |
| Latino | 62 (14.9%) | 14 (22.6%) |
| Other | 24 (5.8%) | 10 (41.7%) |
| Unknown | 76 (18.3%) | 22 (29.0%) |
| Insurance | | p = 0.002 |
| Medicare | 185 (44.6%) | 65 (35.1%) |
| Commercial | 69 (16.6%) | 12 (17.4%) |
| Gov't (Tricare/Colorado | 49 (11.8%) | 18 (36.7%) |
| Access) | | |
| Medicaid/Low-income | 59 (14.2%) | 9 (15.3%) |
| University managed care plan | 53 (12.8%) | 21 (39.6%) |

The proportion who completed such visits arranged by an outreach coordinator was 30.1% (n=125) and the proportion of these who completed laboratory testing at least a day before the provider visit was 80.8% (n=101). In adjusted analyses, men were more likely than women (OR=1.6, 95% CI=1.1-2.5) and patients with Medicaid/low-income health insurance were less likely than those with Medicare to complete a provider visit (OR=0.4, 95% CI=0.2-0.9). After invitation letters were mailed, 65 patients bypassed an outreach coordinator to complete a provider visit that they self-scheduled directly through the clinic. Based on chart review, providers documented attention to diabetes during 80% of these visits (n=52). After including these additional patients who self-scheduled for apparently diabetes-related reasons, the proportion due for provider visits that directly or indirectly responded to outreach was as high as 42.7%, although an unknowable percentage would have scheduled an appointment regardless of receiving an outreach letter. Of the 57.3% that did not complete a provider visit, approximately one-third could not be reached by the outreach coordinator and two-thirds refused for a variety of reasons (see FIG. 30).

### Intervention and Traditional Care Comparisons:

Based on a random chart review of 50 patients who completed a provider visit arranged through outreach, the average number of days was 278 (9.1 months) between this visit and the first preceding visit during which diabetes care was documented in the medical record. By comparison, the elapsed time between the historically most-recent overdue diabetes-related provider visit and the preceding one was 266 days, a non-significant difference (p=0.7). The average number of laboratories due at outreach and traditional care visits was 3.4 and 2.9, respectively, also a non-significant difference (p=0.2). Patient adherence with recommended laboratory testing, referrals for retinal exams, any provider comment related to diabetes, and any combination of diabetes education, endocrinologist consultations, and/or modification of pharmacologic therapy were all significantly more common during provider visits arranged through outreach compared with traditional care (Table 8).

TABLE 8
Completion of services and documentation of
diabetes care: outreach vs. traditional care*

| | Completed | Completed | | |
|---|---|---|---|---|
| | through | through | | |
| | outreach | traditional | | |
| N = 50 patients | (%) | care (%) | Difference | p |
| A1c | 100% | 68% | 32% | <0.001 |
| Lipid | 100% | 58% | 42% | <0.001 |
| Creatinine | 100% | 83% | 17% | 0.02 |
| Microalbumin | 97% | 26% | 73% | <0.001 |
| Eye^{†} | 91% | 23% | 68% | <0.001 |
| Document any | 98% | 74% | 24% | 0.002 |
| attention to diabetes | | | | |
| Refer for diabetes | 48% | 20% | 28% | 0.007 |
| education or modify | | | | |
| pharmacologic | | | | |
| therapy^{‡} | | | | |

*Based on a 50 patient random chart review of 125 patients who completed a provider visit arranged through outreach. Shown are proportions of patients due for indicated services who completed them except that diabetes education referrals and modification of pharmacologic therapy reflect frequency of documentation in the medical record without reference to whether these were due.
^{†}Eye exam was scheduled or patient was instructed to complete.
^{‡}Modification of pharmacologic therapy includes additions or changes to antihyperglycemic medications, blood pressure and lipid-lowering agents, and aspirin.

### Provider Feedback:

Twenty-eight of 31 providers shared their opinions about the intervention through an anonymous online survey. The vast majority of provider attitudes were favorable. Illustrative comments included: “Patients have been pleased with the intervention,” “I think the intervention helps build a relationship with my patients because they know we care about their health even between MD visits,” “It is terrific; we need more of these automatic items,” “Overall, an excellent option. It helps ‘fill in the gaps’ when there are things that just can't be covered in a routine visit.” A small number of providers did report patient concerns. For example, one provider remarked, “I have had at least 2 patients tell me they would not want anyone other than me ordering their tests, and they refused on the phone and then wanted to get my opinion before going through with [the outreach].” Anecdotally, the outreach coordinator reported that many patients appreciated that providers were “keeping track” of their health between visits and that the process of scheduling diabetes services was so convenient.

### Conclusions.

Among patients overdue for recommended services, and in comparison with traditional care, an enhanced reminder/recall intervention was associated with a substantially greater level of concordance with ADA-recommended intervals for diabetes-related laboratories and retinal exam referrals and with more intensive diabetes management during primary care visits. Over 40% of patients completed a diabetes-focused provider visit when recommended in writing or over the phone by an outreach coordinator, and over 80% of those scheduling a provider visit through an outreach coordinator completed laboratories several days in advance of this visit. Only administrative claims data were used to identify eligible patients and an outreach coordinator with a bachelor's degree, rather than a nurse, carried out all tasks related to patient communication, scheduling, and provider notification. Finally, providers expressed a high degree of satisfaction and interest in the intervention.

This unique outreach program is likely to have reduced several barriers associated with traditional care. First, we proactively contacted patients to inform them about recommendations for care instead of waiting for them to make appointments haphazardly and often for reasons unrelated to diabetes. We made daytime and evening telephone calls to patients, many of whom were undoubtedly busy or forgetful, when they did not themselves call in response to letters that summarized personalized recommendations. We scheduled all laboratories and provider visits at a single point in time, over the phone, and then sent reminder postcards that summarized this information. Rather than completing laboratory testing during provider visits, which would have required providers to convey recommendations for care several days later, we scheduled laboratories ahead of time whenever possible so that timely information would be available help guide clinical decision-making at the point of care. Finally, we notified providers in advance about the diabetes-specific purpose of appointments scheduled through the outreach coordinator.

Men were somewhat more responsive to outreach than women. Because women in general were more likely than men to be up to date with provider visits, those who were not may represent a group that is more refractory to outreach. Patients with Medicaid and other low-income forms of insurance were less responsive to outreach than those with other health plans. This substantiates in actual practice the results of a patient survey in which socioeconomically disadvantaged patients were more reticent than other groups about the concept of health promotion outreach (Denberg, T D et al. (2007). It also mirrors the results of a similar intervention designed to promote bone densitometry testing in average-risk women (Example 1). It is possible that socially vulnerable patients misunderstand or distrust the purpose of the intervention or are less able to respond to outreach because of greater numbers of practical barriers (e.g. transportation difficulties, taking time off from work, and child care). On the other hand, individuals with commercial health plans also appear to have been less responsive to outreach than other groups, perhaps because they are more likely to be employed and have difficulty taking time off from work.

Although more than 50% of patients were unresponsive to the outreach program, a large number cited reasons that reflect modifiable misconceptions or misunderstandings about the program or about diabetes. For example, direct provider endorsement and improved education might be beneficial for many patients who do not understand the purpose and mechanics of the outreach program or who downplay the significance of their diabetes.

The chronic care model elaborates key mechanisms for improving the care of patients with diabetes and other chronic illnesses (Bodenheimer T, et al. (2002); Rothman A A, and Wagner E H. (2003); Wagner E H, et al. (1996)) Austin B T, Von Korff M. Organizing care for patients with chronic illness. Milbank Q 1996)).

Our intervention facilitates guideline-based care and promotes chronic care model elements through patient outreach. It offers a powerful mechanism for improving information flow between providers and patients. Continuous outreach also produces more accurate and up-to-date population registries (e.g., identifying patients who die, leave the healthcare system, move, and change phone numbers) than is possible without outreach. Finally, this type of outreach supports the chronic care model's focus on providing patient-centered care: Davis K, et al. (2005)) through outreach, and in anticipation of provider visits, our outreach program provides additional channels for informing and educating patients; it streamlines access to care; and it results in better visit planning and coordination of care.

This study has several weaknesses. Because the outreach program operated in a single academic primary care practice, generalizability is limited. We evaluated only process of care measures, not intermediate or hard clinical outcomes (e.g., changes in hemoglobin A1c values or the development of retinopathy). Based on prior work, outreach and decision support systems that are process-based only can increase the cost of care without improving clinical outcomes, whereas systems that focus on clinical outcomes are more likely to decrease the overall cost of care (Gilmer T P, et al. (2006)). With relatively little effort, however, the intervention can be enhanced and the evaluation period prolonged in order to more directly address clinical outcomes. Because the study was designed in collaboration with our hospital to evaluate the benefits of a new approach to chronic disease care, operational needs precluded a randomized, controlled trial. A pre/post evaluation of outcomes, with patients serving as their own controls, could have potentially produced biased results if other improvements in diabetes care were occurring in the practice at the same time as this intervention, however this was not the case. Finally, the follow-up period was only three months. Despite these limitations, the primary aims of the study were achieved, including assessing the operational feasibility and improvements in care associated with an outreach intervention, gaining insights about the responsiveness of patients to outreach, and evaluating provider interest and satisfaction in the pilot program.

Improvements to the procedures described above include enhancing our administrative claims-based diabetes program to include biometric, laboratory, and medication information culled from the electronic medical record and expanding outreach to patients who are not meeting ADA goals for blood pressure, lipids, and aspirin therapy, as well as facilitating patient access to diabetes educators who are based within our endocrinology practice to sustain improved clinical outcomes for a larger proportion of our diabetes patients.

### Example 3

### Screening Colonoscopy Through Telephone Outreach Without Antecedent Provider Visits

Open access colonoscopy allows relatively healthy patients to receive recommended colorectal cancer (CRC) screening without completing a gastroenterology consultation ahead of time. A logical extension of this idea is to allow patients to obtain screening colonoscopy without requiring that they obtain a referral for this procedure through a face-to-face primary care provider (PCP) visit. We pilot-tested a health promotion outreach system (HPOS) designed to overcome health system and patient barriers to CRC screening (Denberg et al., 2008), including lack of patient knowledge about the benefits of screening, lack of patient awareness about eligibility to complete screening, PCP challenges related to identifying eligible patients during time-limited and often haphazard clinic visits, and a requirement that patients see PCPs ahead of time in order to arrange for screening.

Six PCPs in a large academic general medicine practice participated in this pilot study. Patients of these PCPs were included in a CRC screening registry if they were 50-74 years of age and if, within our billing claims system, they had a record of at least two PCP visits (the last within the previous 18 months) and did not appear to be up to date with CRC screening based on the absence of records indicating completion of a colonoscopy within the past ten years, flexible sigmoidoscopy or double contrast barium enema within the past five years, and fecal occult blood testing (FOBT) within the past year. We excluded patients with significant comorbidities (history of colorectal adenocarcinoma, colectomy or ileostomy, an active cancer or terminal diagnosis, prosthetic heart valve(s), morbid obesity (over 250 pounds), respiratory disease requiring the use of oxygen, unstable angina, end-stage renal disease, and ongoing use of warfarin, heparin, or insulin). Randomly, we selected 125 patients in the screening registry for outreach.

An outreach coordinator with a bachelor's degree was responsible for the day-to-day operation of the intervention. Patient registry records were imported into an information management utility designed for this program. Because colonoscopy is a high-intensity procedure involving more than minimal risk, we sent to each participating PCP an electronic request (Allscripts Touchworks v. 10, Chicago, Ill.) to confirm individual patient eligibility for colonoscopy or an alternative CRC screening test. PCPs returned their responses electronically to the outreach coordinator.

For eligible patients, our information management utility generated invitation letters bearing the printed name of the PCP on the signature line, described the patient's apparent eligibility for CRC screening, summarized the benefits of screening, and recommended that the patient complete colonoscopy or, if recommended by the PCP or desired by the patient, an alternative screening test or a face-to-face discussion with the PCP. Each letter was accompanied by an educational, two-sided “Colorectal Cancer: Basic Facts on Screening” brochure (Centers for Disease Control, 2008).

If a patient did not respond to a letter within two weeks by telephone or an enclosed postage-paid postcard, an outreach coordinator made up to four calls to the patient's home at various times of day, identified herself as calling “on behalf of” the patient's PCP, and left a voice message if necessary on the first and fourth attempts. If telephone contact was established, the outreach coordinator reviewed the recommendations for CRC screening, briefly described colonoscopy or the PCP-indicated alternative, and described alternative screening modalities for patients who were interested. For patients wishing to proceed with colonoscopy, the outreach coordinator encouraged patients to contact their insurance companies to determine their out-of-pocket costs for this procedure. If patients accepted CRC screening, the coordinator immediately scheduled the appropriate service while speaking with the patient. In this way, patients were permitted to omit PCP visits ahead of time. If PCPs or patients preferred to have a face-to-face discussion about CRC screening, the outreach coordinator scheduled a primary care appointment over the phone.

If screening was scheduled, the coordinator subsequently tasked an electronic request for the PCP to authorize the indicated service, ensuring that orders originated with the PCP for billing and compliance, that resulting electronic referrals underwent pre-authorization review, that the results were returned directly to the PCP for clinical follow-up, and that the transaction was documented in the electronic medical record. Once insurance pre-authorization was approved, the coordinator mailed reminder postcards to patients noting the date, time, and location of the services. For colonoscopy procedures, the coordinator sent to patients a summary of instructions and a prescription for the bowel preparation.

We conducted intervention outreach over two months (Oct. 1-Nov. 30, 2007) and tracked adherence for an additional two months (the average wait time for colonoscopy was four to six weeks). Out of 125 patients, 118 were confirmed eligible for telephone outreach to promote CRC screening. Among these patients, 29.7% completed CRC screening by means of colonoscopy, and there were no complications associated with the procedure. No other form of CRC screening was utilized, and all colonoscopy insurance pre-authorization requests were approved. The two most common reasons patients refused to participate included a lack of interest or readiness in screening and a preference to discuss screening with the PCP at an indefinite, future clinic appointment.

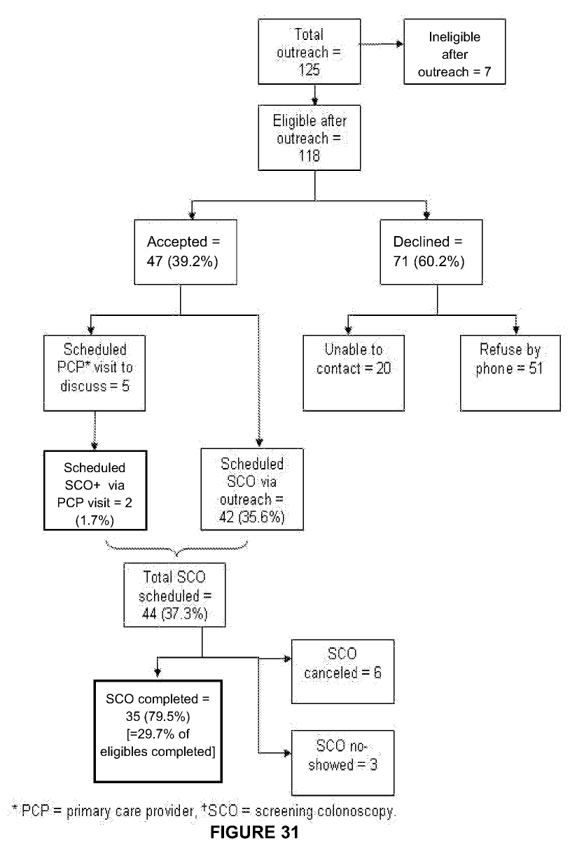

FIG. 31 illustrates numbers and proportions of patients who accepted, declined, completed and cancelled or no-showed for screening colonoscopy. The intervention-related colonoscopy no-show and cancellation rates were 6% and 14%, respectively, which was similar to 5% and 25% identified through usual care. PCPs requested that five patients discuss screening in-person with them before receiving a referral. Patient characteristics (e.g. age, race/ethnicity, marital status) were unassociated with responsiveness to outreach, likely because the total sample size and number of individuals within several demographic categories were relatively small.

All six participating PCPs completed an online survey and reported that they were “very satisfied” with the intervention system, finding the electronic prompts straightforward and easy to manage. On average, it took one minute or less to confirm individual patient eligibility for outreach.

To our knowledge, this is the first time that colonoscopy (a high intensity procedure) has been systematically offered to patients without requiring that they complete PCP visits ahead of time to obtain referrals. We were able to demonstrate that our HPOS intervention is operationally feasible, acceptable to patients and providers, and leads to completion of screening colonoscopy among a substantial proportion of patients who are offered screening. We believe that improved quality of care and performance are likely to result when non-physician team members facilitate the provision of routine preventive services (Yarnell et al., 2003). Our method for promoting CRC screening warrants evaluation within larger, more diverse patient populations, in other health care settings, and with an eye to determining the incremental improvement in screening uptake compared with usual care as well as the costs and adverse events associated with this method of screening promotion.

Impact on physician reimbursement: Assuming that costs per patient encounter are known and that reimbursement/payment for each encounter is at least as great as the value of resources and opportunity costs for that visit, physicians increase profits by increasing patient visits. If a clinic is operating at capacity, the “best” economic strategy for scheduling would be to increase the number of physician visits for types of care (e.g. diabetes-focused) that are the most profitable (e.g., have the greatest difference between payment and economic costs). In addition to economic profitability, it is also in the best interest of clinics to provide care that enhances reputation, such as through better outcomes or quality. Reputation effects can lead to increased demand for services. Therefore, the intervention improves financial performance of the physician group through either increasing the number of care encounters with existing patients, or improving the clinic reputation for enhanced diabetic care such that more patients seek care in these clinics.

Impact on hospital finances. Using the HPO system described herein, the hospital will financially benefit from better compliance with clinic encounters through the provision of ancillary services to the clinic. However, better compliance by patients and improved clinical outcomes also decrease the demand for inpatient care services, emergency department (ED) visits, and other care-related consequences of poor diabetes management, especially for higher-risk patients. Therefore, the impact of the intervention on hospital services results in more revenues from ancillary services to clinics for the intervention patients (vs. usual care) and less provision of inpatient and emergency care for the intervention patients (vs. usual care). Total hospital revenues from the intervention group are less than revenues from the usual care group, since inpatient and emergency care are both relatively costly.

Financial impact for patients: Patients with a given medical condition requiring monitoring have more medical encounters during a given year than similar patients without the condition since it is a condition that requires adjustment of care to ensure reduced risk of adverse health effects in the future. However, most such patients are faced with real economic costs in seeking this recommended care. There are time costs (or opportunity costs) related to scheduling and attending care appointments that might be coordinated poorly such that the patient must make time for multiple phone calls and multiple clinic visits. Most patients also face out-of-pocket costs for coinsurance and co-payments. These financial effects can pose barriers to compliance. The intervention system described herein reduces the barriers related to scheduling time and convenience of appointments, but we are uncertain whether participation will have increased costs to patients. Some patients are good at self-management and schedule appointments and comply with clinical recommendations in the absence of the intervention, so this subgroup can actually see a cost-savings through a more efficient scheduling system, but may not experience a reduction in their out-of-pocket costs. For other patients who are generally not motivated to seek recommended care, the intervention successfully motivates them to schedule more clinic visits and the financial impact is substantial, in terms of increasing out-of-pocket costs for visits. However, their long-term financial benefit is based on a diminished need for costly outpatient care related to new diabetes-related complications as well as less inpatient or emergency care due to poorly managed diabetes. For other patients who are not influenced to increase clinic encounters due to the intervention, short-term and downstream financial impact of the intervention is negligible; they will have minimal costs during the intervention, but risk having increased need for inpatient and other costly care for their conditions in the future.

Financial impact on payers: Payers' incentives are aligned with that of both the clinics and patients. Insurers would like to avoid paying for excessive or unneeded care, but have an incentive to avoid care in more expensive contexts by covering costs for care that can be delivered in a less-expensive setting. For payers who serve a fairly fixed population in terms of demographic and clinical characteristics, preventive and routine care is viewed as a good investment. For payers who serve a patient base that changes frequently, this intervention system can reduce payments for care in more-expensive settings (hospital/emergency rooms) compared usual care.

We have described a unique quality improvement system (HPOS) that is patient-centered, systems-based, population- and disease-focused, and does not require much infrastructure. For primary prevention, HPOS represents an evolutionary step from a simple mailed reminder system to a more personalized, population management system that allows highly-trained clinicians to focus on the more complex aspects of patient care. For chronic disease, HPOS outreach complements and extend the Chronic Care Model (CCM) beyond traditional patient clinic visits. Like the CCM, HPOS is physically based in provider organizations and therefore has many advantages over third party disease management programs. HPOS can also serve as a bridge between traditional practice and the type of wholesale clinic redesign usually contemplated by the CCM. Alleviating some of the production pressures placed on ambulatory care providers by assuming responsibilities that do not require advanced clinical training (e.g. phone outreach), HPOS allows providers to give greater attention to the complexities of diagnosing and treating disease.

HPOS is a novel way of coordinating preventive and chronic disease services for patients in primary care and subspecialty ambulatory practices. It is scalable, adaptable, and economical. It is a powerful tool for improving quality of care and pay-for-performance measures. With the right balance of services for a particular payer mix, it improves guideline-concordant care while increasing clinical revenue. It increases patient satisfaction and allegiance to a health care organization by making best practices transparent and customized to the patient and by eliminating barriers to receiving such care. Consequently, HPOS also provides unique marketing opportunities in a competitive healthcare environment.

The system is useful for programs in both primary care and subspecialty ambulatory practices for chronic obstructive pulmonary disease, congestive heart failure, inflammatory bowel disease, and enhanced diabetes outreach to promote higher rates of concordance with blood pressure goals as well as aspirin and statin therapy. It can also be extended to additional primary prevention services, including yearly influenza vaccinations, blood pressure assessments for the general population, PCP visits to address inadequately-treated hypertension, and patient-informed decision-making around prostate cancer screening.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

### REFERENCES

American Diabetes Association. Standards of medical care in diabetes—2007. Diabetes Care 2007;30 Suppl 1:S4-S41.
Bodenheimer T, Lorig K, Holman H, Grumbach K. Patient self-management of chronic disease in primary care. Jama 2002;288:2469-75.
Bodenheimer, T., Lorig, K., Holman, H., & Grumbach, K. (2002). Patient self-management of chronic disease in primary care. Jama, 288(19), 2469-2475.
Bodenheimer, T., Wagner, E. H., & Grumbach, K. (2002). Improving primary care for patients with chronic illness. Jama, 288(14), 1775-1779.
Centers for Disease Control. (2008). Colorectal Cancer: Basic Facts about Screening (brochure). CDC website.
Davis K, Schoenbaum S C, Audet A M. A 2020 vision of patient-centered primary care. J Gen Intern Med 2005;20:953-7.
Denberg T D, Lin C T, Myers B A, Cashman J M, Kutner J S, Steiner J F. Improving Patient Care Through Health-Promotion Outreach. J. Ambulatory Care Management 2008;31:54-65.
Denberg T D, Myers B A, Lin C T, Libby A M, Min S-J, McDermott M T, et al. An outreach intervention increases bone densitometry testing in older women. In press, J. American Geriatrics Society; 2008.
Denberg T D, Ross S E, Steiner J F. Patient acceptance of a novel preventive care delivery system. Prev Med 2007;44:543-6.
Denberg, T. D., Coombes, J. M., Byers, T. E., Marcus, A. C., Feinberg, L. E., Steiner, J. F., et al. (2006). Effect of a mailed brochure on appointment-keeping for screening colonoscopy: a randomized trial. Ann Intern Med, 145(12), 895-900.
Disease Management Association of America (DMAA) website. Definition of Disease Management.
Dorr, D. A., Wilcox, A., McConnell, K. J., Burns, L., & Brunker, C. P. (2007). Productivity enhancement for primary care providers using multicondition care management. Am J Manag Care, 13(1), 22-28.
Fitzner, K., Fox, K., Schmidt, J., Roberts, M., Rindress, D., & Hay, J. (2005). Implementation and outcomes of commercial disease management programs in the United States: the disease management outcomes consolidation survey. Dis Manag, 8(4), 253-264.
Gilmer T P, O'Connor P J, Rush W A, Crain A L, Whitebird R R, Hanson A M, et al. Impact of office systems and improvement strategies on costs of care for adults with diabetes. Diabetes Care 2006;29:1242-8.
Glasgow, R. E., Funnell, M. M., Bonomi, A. E., Davis, C., Beckham, V., & Wagner, E. H. (2002). Self-management aspects of the improving chronic illness care breakthrough series: implementation with diabetes and heart failure teams. Ann Behav Med, 24(2), 80-87.
Glasgow, R. E., Orleans, C. T., & Wagner, E. H. (2001). Does the chronic care model serve also as a template for improving prevention? Milbank Q, 79(4), 579-612, iv-v.
Hosmer D W, Lemeshow S. Applied Logistic Regression. New York: Wiley; 2000.
Kabcenell, A., Langley, J., & Hupke, C. (2006 (Available on IHI.org website)). Innovations in Planned Care. Cambridge, Mass.: Institute for Healthcare Improvement.
Kerr, E. A., Gerzoff, R. B., Krein, S. L., Selby, J. V., Piette, J. D., Curb, J. D., et al. (2004). Diabetes care quality in the Veterans Affairs Health Care System and commercial managed care: the TRIAD study. Ann Intern Med, 141(4), 272-281.
Lomas, J. (2003). Health services research. Bmj, 327(7427), 1301-1302.
Nutting, P. A., Dickinson, W. P., Dickinson, L. M., Nelson, C. C., King, D. K., Crabtree, B. F., et al. (2007). Use of chronic care model elements is associated with higher-quality care for diabetes. Ann Fam Med, 5(1), 14-20.
Ostbye T, Yarnall K S, Krause K M, Pollak K I, Gradison M, Michener J L. Is there time for management of patients with chronic diseases in primary care? Ann Fam Med 2005;3:209-14.
Ouwens, M., Wollersheim, H., Hermens, R., Hulscher, M., & Grol, R. (2005). Integrated care programmes for chronically ill patients: a review of systematic reviews. Int J Qual Health Care, 17(2), 141-146.
Rothman A A, Wagner E H. Chronic illness management: what is the role of primary care? Ann Intern Med 2003;138:256-61.
Schmittdiel, J., McMenamin, S. B., Halpin, H. A., Gillies, R. R., Bodenheimer, T., Shortell, S. M., et al. (2004). The use of patient and physician reminders for preventive services: results from a National Study of Physician Organizations. Prev Med, 39(5), 1000-1006.
St Peter, R. F., Reed, M. C., Kemper, P., & Blumenthal, D. (1999). Changes in the scope of care provided by primary care physicians. N Engl J Med, 341(26), 1980-1985.
Stevens, N. H. (1998). KPNW's Safety Net for Preventive Services: The Challenge of Reaching the Unscreened. The Permanente Journal, 2(4), 54-61.
Wagner E H, Austin B T, Von Korff M. Organizing care for patients with chronic illness. Milbank Q 1996;74:511-44.
Wallace, P. J. (2005). Physician involvement in disease management as part of the CCM. Health Care Financ Rev, 27(1), 19-31.
Yarnall K S, Pollak K I, Ostbye T, Krause K M, Michener J L. Primary care: is there enough time for prevention? Am J Public Health 2003;93:635-41.

1. A health promotion outreach (HPO) system for increasing access of patients to medical services performed by a medical facility, said system comprising:
a computer processor comprising an electronic patient registry; and
a tracking algorithm with which said processor is programmed, said tracking algorithm being operationally linked with said patient registry, said tracking algorithm comprising:
means for electronically accessing selected data from patient medical and administrative records of said medical facility and populating said patient registry with said selected data;
means for recording, sorting and displaying data in said patient registry; and
means for recording, sorting and displaying patient status in an HPO process, wherein said HPO process comprises:
identifying and confirming eligibility of patients of said medical facility for selected medical services;
contacting patients who are confirmed to be eligible for said selected medical services, said patients being physically located outside said medical facility;
scheduling said selected medical services for said patients; and
recording results of said selected medical services.

2. The HPO system of claim 1 also comprising electronic means operationally linked to said tracking algorithm for tracking confirmation of eligibility of patients for selected medical services by recording fulfillment of specified clinical criteria and/or authorization from medical providers to perform said medical services.

3. The HPO system of claim 1 also comprising electronic means operationally linked to said tracking algorithm for tracking confirmation of eligibility of patients for selected medical services by recording authorization from insurance payers for said patients to receive said selected medical services.

4. The HPO system of claim 1 also comprising electronic means operationally linked to said tracking algorithm for generating written communications with patients confirmed as eligible for said selected medical services asking them to contact a representative of said HPO system for scheduling of said selected medical services.

5. The HPO system of claim 4 also comprising electronic means operationally linked to said tracking algorithm for inputting, recording and displaying results of said written communications.

6. The HPO system of claim 1 also comprising electronic means operationally linked to said tracking algorithm for identifying and displaying contact information for patients requiring telephone contact.

7. The HPO system of claim 6 also comprising electronic means operationally linked to said tracking algorithm for inputting, recording and displaying results of telephone contact with patients.

8. The HPO system of claim 1 also comprising electronic means operationally linked to said tracking algorithm for scheduling appointments or recording information related to scheduling appointments for medical services for eligible patients.

9. The HPO system of claim 1 also comprising electronic means operationally linked to said tracking algorithm for generating written reminders to patients of scheduled appointments.

10. The HPO system of claim 1 also comprising electronic means operationally linked to said tracking algorithm for recording results of said scheduled appointments.

11. The HPO system of claim 1 also comprising electronic means operationally linked to said tracking algorithm for updating said patient registry.

12. The HPO system of claim 1 also comprising electronic means operationally linked to said tracking algorithm for generating reports utilizing data stored in said system.

13. The HPO system of claim 10 wherein said reports comprise selected combinations of types of information stored in said processor, said types of information being selected from the group consisting of medical service, eligibility for service, medical provider, medical facility, test results, patient appointment, patient compliance, number and type of patient contacts, service dates, patient demographics.

14. A computer-implemented Health Promotion Outreach (HPO) process for increasing patient access to, and enhancing delivery of, medical services performed by a medical facility, wherein the position of patients within said process are tracked in an HPO system of claim 1; and wherein said HPO process does not require patients to personally see their medical providers as a prerequisite for scheduling and performing of said medical services; said process comprising:
a computer-implemented step of identifying and confirming eligibility of patients of said medical facility for selected medical services;
a computer-implemented step of contacting patients who are confirmed to be eligible for said selected medical services, said patients being physically located outside said medical facility;
a computer-implemented step of scheduling said selected medical services for said patients; and
a computer-implemented step of recording results of said selected medical services.

15. The HPO process of claim 14 also comprising a computer-implemented step of obtaining and recording authorizations from medical providers and fulfillment of clinical criteria to perform said medical services, and electronically storing a record of said authorizations in said processor such that they are accessible to said tracking algorithm.

16. The HPO process of claim 14 also comprising a computer-implemented step of obtaining and recording authorizations from insurance payers for said patients to perform said selected medical services, and electronically storing a record of said authorizations in said processor such that they are accessible to said tracking algorithm.

17. The HPO process of claim 14 also comprising a computer-implemented step of generating written communications with patients confirmed as eligible for said selected medical services asking said patients to contact a representative of said HPO system for scheduling of said selected medical services, transmitting said written communications to said patients, and electronically storing a record of said generated written communications in said processor such that they are accessible to said tracking algorithm.

18. The HPO process of claim 14 also electronically storing a record of results of said written communications in said processor such that they are accessible to said tracking algorithm.

19. The HPO process of claim 14 also comprising causing said processor to display contact information for patients requiring telephone contact.

20. The HPO process of claim 17 also comprising a computer-implemented step of making telephone contact with said patients requiring telephone contact and electronically storing a record of said telephone contacts in said processor such that they are accessible to said tracking algorithm.

21. The HPO process of claim 14 also comprising a computer-implemented step of scheduling appointments for medical services for eligible patients and electronically storing a record of said appointments in said processor such that they are accessible to said tracking algorithm.

22. The HPO process of claim 19 also comprising a computer-implemented step of generating written reminders to patients of said scheduled appointments, transmitting said written reminders to said patients, and electronically storing a record of said generated written reminders in said processor such that they are accessible to said tracking algorithm.

23. The HPO process of claim 14 also comprising electronically recording results of medical services performed for patients in said processor such that they are accessible to said tracking algorithm.

24. The HPO process of claim 14 also comprising electronically generating reports utilizing data stored in said system.

25. The HPO process of claim 14 wherein said medical services are guideline-concordant medical services.

26. The HPO process of claim 14 wherein said guideline-concordant medical services are selected from the group consisting of: chronic disease maintenance and preventive screening services.

27. The HPO process of claim 24 wherein said medical services are selected from the group consisting of one or more services pertinent to one or more chronic diseases and preventive services.

28. The HPO process of claim 27 wherein said preventive services are selected from the group consisting of immunizations, behavioral risk assessments, and screening tests for cancer, endocrine, pulmonary, gastrointestinal, psychiatric, developmental, musculoskeletal, neurological, genitourinary, and cardiovascular disease.
